(19) United States
(12) Patent Application Publication
(10) Pub. No.: US 2025/0171391 A1
(43) Pub. Date: May 29, 2025

(54) PROCESS FOR PREPARING OCTAHYDRO-2(1H)-NAPHTHALENONE DERIVATIVES

(71) Applicant: Firmenich SA, Satigny (CH)

(72) Inventors: Oliver KNOPFF, Satigny (CH); Nicolas POIRIER, Satigny (CH); Murielle HALDIMANN SANCHEZ, Satigny (CH); Virginie BLANCHARD, Satigny (CH)

(21) Appl. No.: 18/834,450

(22) PCT Filed: Feb. 15, 2023
(86) PCT No.: PCT/EP23/53712

§ 371 Date: Jul. 30, 2024

(30) Foreign Application Priority Data
Feb. 16, 2022 (EP) 22156988.2

(51) Int. Cl. C07C 45/40; C07C 67/44; C07D 323/02

(52) U.S. Cl. CPC C07C 45/40; C07C 67/44; C07D 323/02; C07B 2200/07

(57) ABSTRACT

Disclosed herein is a process for preparing a compound of formula (I). Also disclosed herein are compounds of formula (V), formula (VI), and formula (VIII).

### TECHNICAL FIELD

The present invention relates to the field of organic synthesis and more specifically it concerns a process for preparing compound of formula (I). The compound of formula (V), the compound of formula (VI) and the compound of formula (VIII) are also part of the invention.

### BACKGROUND OF THE INVENTION

The octahydro-2(1H)-naphthalenone derivatives represent skeletons highly desirables which could be used as such or as key intermediates useful to prepare more complex compounds in different fields such as, among others, perfumery, cosmetic, pharmaceutic or agrochemistry. Relevant octahydro-2(1H)-naphthalenone derivatives in perfumery industry are, for example, 5,5,8a-trimethyloctahydro-2(1H)-naphthalenone which is a valuable intermediate towards 5,5,8a-trimethyldecahydronaphthalen-2-yl acetate representing some of the most sought-after ingredients in the perfumery industry. Both compounds possess several stereocenters and may be in the form of different stereoisomers wherein the perfuming interest is particularly directed toward stereoisomers having a trans decaline. The preparation of compound of formula (I) with high selectivity toward the trans decaline, starting from commercially available geraniol, has been reported in EP579991 and requests eight chemical steps. Alternatively, WO2020173977 discloses the preparation of compound of formula (I) in a high selectivity via the cyclisation of 6,10-dimethylundeca-1,5,9-triene or 6,10-dimethylundeca-5,9-dien-1-yne in the presence of squalene hopene cyclase. However, the conversion and yield are low, the reaction time is very long and the preparation of both starting materials are not industrially applicable.

Being products of industrial interest, there is always a need for new processes showing an improved yield or productivity while limiting the number of steps without compromising the selectivity. Recently, biotechnological methods to produce ((4aSR,8aSR)-5,5,8a-trimethyl-2-methylenedecahydronaphthalen-1-yl)methanol or ((4aSR,8aSR)-5,5,8a-trimethyl-2-methylenedecahydronaphthalen-1-yl)methyl acetate in high selectivity using host cells/microorganisms expressing relevant enzymes have been reported in WO2018220113, WO2019229064 or WO2020078871. However, the conversion of those compounds into compound of formula (I) has never been reported.

The present invention is a process for preparing compound of formula (I) with high selectivity towards the trans decaline isomer, starting from compound of formula (II) via a novel route through novel intermediates. In particular, the compounds of formula (V), (VI) and (VIII) which are an object of the present invention, have never been reported in the prior art or suggested in the context of the preparation of compounds of formula (I). To the best of our knowledge, the invention's process or the invention's compounds of formula (V), (VI) and (VIII) have never been reported in the prior art.

### SUMMARY OF THE INVENTION

The invention relates to a novel process allowing the preparation of compound of formula (I) starting from compound of formula (II) opening a new route towards compound of formula (IV).

So, the first object of the present invention is a process the preparation of a compound of formula

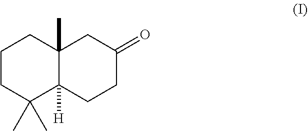

wherein the bold and hatched lines indicate a relative or absolute configuration; comprising the steps of
   a) oxidative cleavage of compound of formula

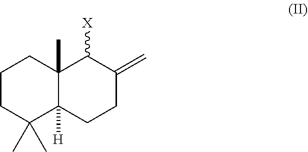

in the form of any one of its stereoisomers or a mixture thereof, and wherein X represents a vinyl, a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═O)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group; or one R^{c} and R^{d}, when taken together, form a C₄₋₅ oxacycloalkyl group;
   into an intermediate of formula

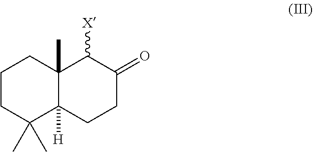

in the form of any one of its stereoisomers or a mixture thereof, and wherein
X′ represents a CHO, a COOH, a C(═O)OR¹ or a CH₂OR² group and R¹ and
R² have the same meaning as defined above; and
   b) the conversion of the intermediate of formula (III) to the compound of formula (I).

A second object of the present invention is a compound of formula

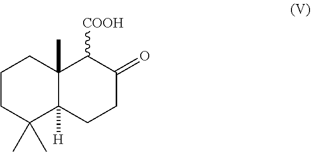 [SMILES: CC1(C)CCC[C@]2(C)C(C(=O)O)C(=O)CC[C@@H]12]

wherein the bold and hatched lines indicate a relative or absolute configuration.

A third object of the present invention is a compound of formula

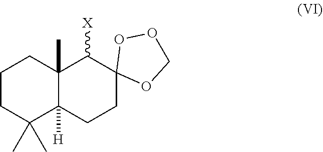

in the form of any one of its stereoisomers or a mixture thereof, wherein the bold and hatched lines indicate a relative or absolute configuration; X represents a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═O)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group or one R^{c} and R^{d}, when taken together, form a C₄₋₅ oxacycloalkyl group.

A further object of the present invention is a compound of formula

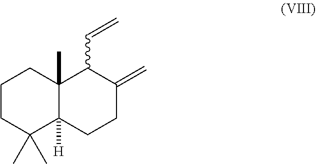 [SMILES: C=CC1C(=C)CC[C@H]2C(C)(C)CCC[C@]12C]

wherein the bold and hatched lines indicate a relative or absolute configuration.

### DESCRIPTION OF THE INVENTION

It has now been surprisingly found that compound of formula (I) with a trans configuration may be obtained from compound of formula (II) allowing to reduce the number of steps while maintaining a high selectivity and yield. The invention's process opens a new route starting from a natural product or derivatives from the natural product allowing obtaining compound of formula (IV) with overall higher yield, compared to the methods known from the prior arts and without formation of the cis decaline.

So, the first object of the invention is a process for the preparation of a compound of formula

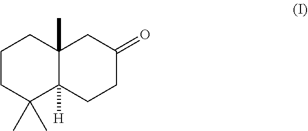

wherein the bold and hatched lines indicate a relative or absolute configuration; comprising the steps of
   a) oxidative cleavage of compound of formula

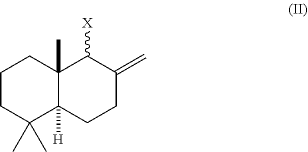

in the form of any one of its stereoisomers or a mixture thereof, and wherein X represents a vinyl, a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group; or one R^{c} and R^{d}, when taken together, form a C₄₋₅ oxacycloalkyl group;
into an intermediate of formula

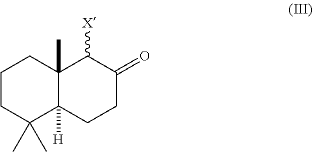

in the form of any one of its stereoisomers or a mixture thereof, and wherein
X′ represents a CHO, a COOH, a C(═O)OR¹ or a CH₂OR² group and R¹ and R² have the same meaning as defined above; and
   b) the conversion of the intermediate of formula (III) to the compound of formula (I).

For the sake of clarity, by the expression “the bold and hatched lines indicate a relative or absolute configuration” or the similar, it is meant the normal meaning understood by a person skilled in the art, i.e. that in the case of an relative configuration compound (I) is in the form of a mixture of stereoisomers comprising more than 50% (w/w) of the (4aSR,8aRS) stereoisomer, i.e. a compound having a decaline group in a relative trans configuration as shown in formula (I), or in the case of an absolute configuration compound (I) is in the form of a mixture of stereoisomers comprising more than 50% (w/w) of the (4aS,8R) stereoisomer.

By the term “oxidative cleavage” or similar, it is meant the normal meaning in the art; i.e. a reaction in which a carbon-carbon double bond is cleaved and oxidized generating two compounds having a carbon-oxygen double bond.

The wavy line of compound of formula (II) and (III) indicates that the carbon stereocenter bearing said bond may be in a R or S relative or absolute configuration. In other words, said bond may be in the same side than the methyl group at the ring junction or said bond may be in the opposite side than the methyl group at the ring junction.

For the sake of clarity, by the expression “any one of its stereoisomers or a mixture thereof”, or the similar, it is meant the normal meaning understood by a person skilled in the art, i.e. that the compounds of formula (II) and (III) can be a pure enantiomer or a mixture of enantiomers provided, of course, that the decaline group have a trans configuration. In other words, the compounds of formula (II) and (III) possess three stereocenter which can have two different stereochemistries (e.g. R or S). The compounds of formula (II) and (III) may even be in the form of a pure enantiomer or in the form of a mixture of enantiomers. The compounds of formula (II) and (III) may even be in the form of a pure diastereoisomer or in the form of a mixture of diastereoisomers. The compounds of formula (II) and (III) can be in a racemic form or scalemic form. Therefore, the compounds of formula (II) and (III) can be one stereoisomer or in the form of a composition of matter comprising, or consisting of, various stereoisomers.

The term “alkyl” is understood as comprising branched and linear alkyl groups.

For the sake of clarity, by the expression “one R^{c} and R^{d}, when taken together, form a C₄₋₅ oxacycloalkyl group . . . .”, it is meant that the carbon atom and oxygen atom to which both groups are bonded are included into the C₄₋₅ oxacycloalkyl group.

According to a particular embodiment of the invention, the compound of formula (II) can be a compound of formula

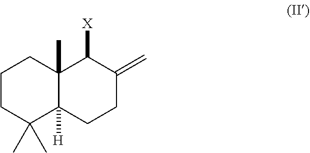

wherein the bold and hatched lines and X have the meaning indicated in formula (I).

According to a particular embodiment of the invention, the compound of formula (II) can be a compound of formula

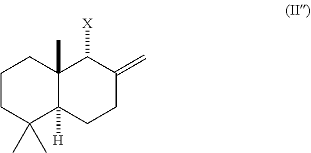

wherein the bold and hatched lines and X have the meaning indicated in formula (I).

According to a particular embodiment of the invention, the compound of formula (II) can be in a form of a composition comprising compound of formula (II′) and compound of formula (II″).

According to a particular embodiment of the invention, the compound of formula (III) can be a compound of formula

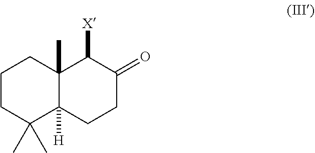

wherein the bold and hatched lines and X′ have the meaning indicated in formula (I).

According to a particular embodiment of the invention, the compound of formula (III) can be a compound of formula

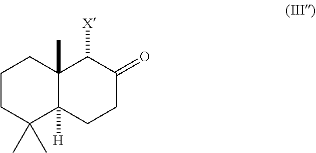

wherein the bold and hatched lines and X′ have the meaning indicated in formula (I).

According to a particular embodiment of the invention, the compound of formula (III) can be in a form of a composition comprising compound of formula (III′) and compound of formula (III″).

According to any embodiment of the invention, R¹ may be a C₁₋₄ alkyl group. Particularly, R¹ may be a C₁₋₃ alkyl group. Even more particularly, R¹ may a methyl group.

According to any embodiment of the invention, R^{a} may be a hydrogen atom or a C₁. 4 alkyl group. Particularly, R^{a} may be a hydrogen atom a C₁₋₃ alkyl group. Particularly, R^{a} may be a hydrogen atom or a methyl or an ethyl group. Even more particularly, R^{a} may a hydrogen atom or a methyl group. Even more particularly, R^{a} may a hydrogen atom.

According to any embodiment of the invention, R^{b} may be a C₁₋₄ alkyl group. Particularly, R^{b} may be a C₁₋₃ alkyl group. Even more particularly, R^{b} may a methyl or an ethyl group.

According to any embodiment of the invention,one R^{c} may be a hydrogen atom or a C₁₋₂ alkyl group and the other R^{c} may be a hydrogen atom; i.e. R² may be a CHR^{c}OR^{d} group. Particularly, one R^{c} may be a hydrogen atom or a methyl group and the other R^{c} may be a hydrogen atom. Even more particularly both R^{c} may be a hydrogen atom; i.e. R² may be a CH₂OR^{d} group.

According to any embodiment of the invention, R^{d} may be a C₁₋₃ alkyl group. Even more particularly, R^{d} may a methyl or an ethyl group.

According to any embodiment of the invention, one R^{c} and R^{d}, when taken together, may form a C₅ oxacycloalkyl group.

According to any embodiment of the invention, R² may be a hydrogen atom or a C(═O)R^{a} group. Particularly, R² may be a hydrogen atom.

According to any embodiment of the invention, X may be a CHO, a COOH, a C(═O)OR¹ or a CH₂OR² group. Particularly, X may be a CHO, a COOH or a CH₂OH group. Even more particularly, X may be a CH₂OH group.

According to any embodiment of the invention, X′ may be a CHO, a COOH or a CH₂OR² group. Particularly, X′ may be a COOH or a CH₂OR² group. Particularly, X′ may be a COOH or a CH₂OH group. Even more particularly, X′ may be a CH₂OH group.

Non limiting examples of compound of formula (II) may include ((4aSR,8aSR)-5,5,8a-trimethyl-2-methylenedecahydronaphthalen-1-yl)methanol, ((4aS,8aS)-5,5,8a-trimethyl-2-methylenedecahydronaphthalen-1-yl)methanol, ((4aR,8aR)-5,5,8a-trimethyl-2-methylenedecahydronaphthalen-1-yl)methanol, (4aSR,8aSR)-5,5,8a-trimethyl-2-methylenedecahydronaphthalene-1-carbaldehyde, (4aS,8aS)-5,5,8a-trimethyl-2-methylenedecahydronaphthalene-1-carbaldehyde, (4aR,8aR)-5,5,8a-trimethyl-2-methylenedecahydronaphthalene-1-carbaldehyde, (4aSR,8aSR)-5,5,8a-trimethyl-2-methylenedecahydronaphthalene-1-carboxylic acid, (4aS,8aS)-5,5,8a-trimethyl-2-methylenedecahydronaphthalene-1-carboxylic acid, (4aR,8aR)-5,5,8a-trimethyl-2-methylenedecahydronaphthalene-1-carboxylic acid, methyl or ethyl (4aSR,8aSR)-5,5,8a-trimethyl-2-methylenedecahydronaphthalene-1-carboxylate, methyl or ethyl (4aS,8aS)-5,5,8a-trimethyl-2-methylenedecahydronaphthalene-1-carboxylate, methyl or ethyl (4aR,8aR)-5,5,8a-trimethyl-2-methylenedecahydronaphthalene-1-carboxylate, ((4aSR,8aSR)-5,5,8a-trimethyl-2-methylenedecahydronaphthalen-1-yl)methyl formate, ((4aS,8aS)-5,5,8a-trimethyl-2-methylenedecahydronaphthalen-1-yl)methyl formate, ((4aR,8aR)-5,5,8a-trimethyl-2-methylenedecahydronaphthalen-1-yl)methyl formate, ((4aSR,8aSR)-5,5,8a-trimethyl-2-methylenedecahydronaphthalen-1-yl)methyl acetate, ((4aS,8aS)-5,5,8a-trimethyl-2-methylenedecahydronaphthalen-1-yl)methyl acetate, ((4aR,8aR)-5,5,8a-trimethyl-2-methylenedecahydronaphthalen-1-yl)methyl acetate, trimethyl(((4aSR,8aSR)-5,5,8a-trimethyl-2-methylenedecahydronaphthalen-1-yl)methoxy)silane, trimethyl(((4aS,8aS)-5,5,8a-trimethyl-2-methylenedecahydronaphthalen-1-yl)methoxy)silane, trimethyl(((4aR,8aR)-5,5,8a-trimethyl-2-methylenedecahydronaphthalen-1-yl)methoxy)silane, (4aSR,8aSR)-5-((1-ethoxyethoxy)methyl)-1,1,4a-trimethyl-6-methylenedecahydronaphthalene, (4aS,8aS)-5-((1-ethoxyethoxy)methyl)-1,1,4a-trimethyl-6-methylenedecahydronaphthalene, (4aR,8aR)-5-((1-ethoxyethoxy)methyl)-1,1,4a-trimethyl-6-methylenedecahydronaphthalene, (4aSR,8aSR)-5-((benzyloxy)methyl)-1,1,4a-trimethyl-6-methylenedecahydronaphthalene, (4aS,8aS)-5-((benzyloxy)methyl)-1,1,4a-trimethyl-6-methylenedecahydronaphthalene, (4aR,8aR)-5-((benzyloxy)methyl)-1,1,4a-trimethyl-6-methylenedecahydronaphthalene, (4aSR,8aSR)-1,1,4a-trimethyl-6-methylene-5-vinyldecahydronaphthalene, (4aS,8aS)-1,1,4a-trimethyl-6-methylene-5-vinyldecahydronaphthalene, (4aR,8aR)-1,1,4a-trimethyl-6-methylene-5-vinyldecahydronaphthalene.

According to any embodiments of the invention the compound of formula (II) may be prepared according to method known by the person skilled in the art. Particularly, compound of formula (II) wherein X is a CH₂OR² group wherein R² represents a hydrogen atom or a C(═O)Me group may be produced in vitro using purified recombinantly prepared enzymes or by fermentation using host cells, such as microbial cells, genetically engineered to convert unexpensive carbon sources (such as sugar) into the desired compound of formula (II), using a Haloacid dehalogenase-like (HAD-like) hydrolase superfamily as reported in WO2018220113 or WO2019229064 and then followed, when X is a CH₂OR² group wherein R² is a C(═O)Me group, by an acetyl transferase as disclosed in WO2020078871. The advantage of using a compound of formula (II) obtained by fermentation is evident since it allows an easy access to the starting material with high selectivity.

According to any one of the above embodiments of the invention's process, said process is further characterized in that the compound of formula (II) is obtained, in a previous step, by contacting farnesyl pyrophosphate with at least one enzyme such as the one reported in WO2018220113 or WO2019229064 and then optionally with the one reported in WO2020078871. Other steps may be needed depending of the nature of the X group, such as protection or oxidation, optionally followed by esterification. Said steps are well known in the art and the person skilled in the art is able to select the most suitable conditions.

Non limiting examples of compound of formula (III) may include (4aSR,8aSR)-1-(hydroxymethyl)-5,5,8a-trimethyloctahydronaphthalen-2(1H)-one, (4aS,8aS)-1-(hydroxymethyl)-5,5,8a-trimethyloctahydronaphthalen-2(1H)-one, (4aSR,8aSR)-5,5,8a-trimethyl-2-oxodecahydronaphthalene-1-carboxylic acid, (4aS,8aS)-5,5,8a-trimethyl-2-oxodecahydronaphthalene-1-carboxylic acid, methyl or ethyl (4aSR,8aSR)-5,5,8a-trimethyl-2-oxodecahydronaphthalene-1-carboxylate or methyl or ethyl (4aS,8aS)-5,5,8a-trimethyl-2-oxodecahydronaphthalene-1-carboxylate, ((4aSR,8aSR)-5,5,8a-trimethyl-2-oxodecahydronaphthalen-1-yl)methyl formate, ((4aS,8aS)-5,5,8a-trimethyl-2-oxodecahydronaphthalen-1-yl)methyl formate, ((4aR,8aR)-5,5,8a-trimethyl-2-oxodecahydronaphthalen-1-yl)methyl formate, ((4aSR,8aSR)-5,5,8a-trimethyl-2-oxodecahydronaphthalen-1-yl)methyl acetate, ((4aS,8aS)-5,5,8a-trimethyl-2-oxodecahydronaphthalen-1-yl)methyl acetate, ((4aR,8aR)-5,5,8a-trimethyl-2-oxodecahydronaphthalen-1-yl)methyl acetate, (4aSR,8aSR)-5,5,8a-trimethyl-1-(((trimethylsilyl)oxy)methyl)octahydronaphthalen-2(1H)-one, (4aS,8aS)-5,5,8a-trimethyl-1-(((trimethylsilyl)oxy)methyl)octahydronaphthalen-2(1H)-one, (4aR,8aR)-5,5,8a-trimethyl-1-(((trimethylsilyl)oxy)methyl)octahydronaphthalen-2(1H)-one, (4aSR,8aSR)-1-((1-ethoxyethoxy)methyl)-5,5,8a-trimethyloctahydronaphthalen-2(1H)-one, (4aS,8aS)-1-((1-ethoxyethoxy)methyl)-5,5,8a-trimethyloctahydronaphthalen-2(1H)-one, (4aR,8aR)-1-((1-ethoxyethoxy)methyl)-5,5,8a-trimethyloctahydronaphthalen-2(1H)-one, (4aSR,8aSR)-1-((benzyloxy)methyl)-5,5,8a-trimethyloctahydronaphthalen-2(1H)-one, (4aS,8aS)-1-((benzyloxy)methyl)-5,5,8a-trimethyloctahydronaphthalen-2(1H)-one, (4aR,8aR)-1-((benzyloxy)methyl)-5,5,8a-trimethyloctahydronaphthalen-2(1H)-one, (4aSR,8aSR,Z)-1-(hydroxymethylene)-5,5,8a-trimethyloctahydronaphthalen-2(1H)-one, (4aS,8aS,Z)-1-(hydroxymethylene)-5,5,8a-trimethyloctahydronaphthalen-2(1H)-one, (4aR,8aR,Z)-1-(hydroxymethylene)-5,5,8a-trimethyloctahydronaphthalen-2(1H)-one.

According to any embodiments of the invention, the oxidative cleavage may be carried out under normal condition known by the person skilled in the art, i.e. in the presence of an oxidizing agent such as ozone, reaction also known as ozonolysis, OsO₄/NaIO₄, KMnO₄/NaIO₄, RuCl₃/NaIO₄, RuCl₃/NaOCl, H₂O₂/NaIO₄ or organic peroxide/NaIO₄. Particularly, the oxidative cleavage may be an ozonolysis; i.e. compound of formula (II) reacts with ozone. Even more particularly, the oxidative cleavage may be an ozonolysis performed under reductive conditions.

For the sake of clarity, by the expression “under reductive conditions” it is understood by a person skilled in the art that the intermediate trioxolane or hydroperoxide formed, to obtain compound of formula (III), is treated with at least one reducing agent, which is well known to a person skilled in the art. Such treatment with a reducing agent can be performed during the work-up. As non-limiting examples of said reducing agents one may cite the following: an amine in particular a tertiary amine or a pyridine, a sulfite, such as an alkaline sulfite (e.g. sodium or potassium sulfite, sodium bisulfite) or a C₂₋₆ dialkyl sulfide such as dimethyl sulfide or methylphenylsulfide, Na salt of 3,3′-Thiodipropionic acid, triphenylphosphine, Zn/AcOH, Zn/AcOH/water, Na₂S, thiourea, thiodiglycol, 3,3′-Thiodipropanol, 3,3′-Thiodipropionitrile, H₂ and Pd/C or Raney/Ni, P(OMe)₃, P(OEt)₃, P(OPh)₃ MeO(SO)OMe, MeSSMe, etc. In particular one may cite a sulfite, such as an alkaline sulfite (e.g. sodium or potassium sulfite, sodium bisulfite) optionally in combination with Na salt of 3,3′-Thiodipropionic acid or a C₂₋₆ dialkyl sulfide such as dimethyl sulfide.

When the oxidative cleavage is carried out with compound of formula (II) wherein X is a CHO group, the oxidative cleavage provided a compound of formula (III) wherein X′ is a COOH group. In other words, the CHO group is oxidized under oxidative cleavage conditions.

When the oxidative cleavage is carried out with compound of formula (II) wherein X is a vinyl group, the oxidative cleavage provided a mixture comprising a compound of formula (III) wherein X′ is a COOH group and a compound of formula (III) wherein X′ is a CHO group. In other words, the vinyl group is partly oxidized under oxidative cleavage conditions. A compound of formula (III) wherein X′ is a CHO may be in a form of a enal of formula (III^{a})

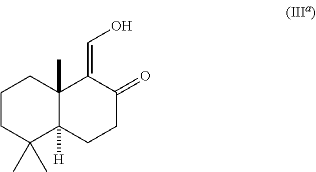 [SMILES: CC1(C)CCC[C@]2(C)/C(=C/O)C(=O)CC[C@@H]12]

wherein the hold and hatched lines indicate a relative or absolute configuration.

The ozonolyis can be carried out in the presence or absence of a solvent. When a solvent is required or used for practical reasons, then any solvent wherein the compound of formula (II) is soluble and which is of current use in ozonolysis reactions can be used for the purposes of the invention. Non-limiting examples include C₆₋₁₀ saturated hydrocarbon solvents such as hexane or cyclohexane, saturated C₄₋₁₀ ethers or esters such as AcOEt, tetrahydrofuran, dioxane or MTBE, saturated C₂₋₅ carboxylic acids such as acidic or propionic acid, saturated C₁₋₅ polar solvents such as primary or secondary alcohols such as isopropanol, methanol or ethanol, saturated C₂₋₆ ketones such as butanone or isobutylmethylketone, C₁₋₃ chlorinated alkane such as chloroform or dichloromethane, or mixtures thereof. The exact choice of the solvent is a function of the compound of formula (II) and reaction speed required. The person skilled in the art is well able to select the solvent most convenient in each case to optimize the ozonolysis reaction.

The solvent can be added to the reaction medium in a large range of concentrations. As non-limiting examples, one can cite as solvent amounts ranging from 50% to 500% w/w, relative to the amount of compound of formula (II) used.

The temperature at which the oxidation can be carried out is comprised between −100° C. and 40° C., particularly, in the range of between −80° C. and 20° C., even more particularly, in the range of between −40° C. and 0° C. Of course, a person skilled in the art is able to select the preferred temperature as a function of the melting and boiling point of the starting and final products as well as the desired time of reaction or conversion.

The ozone can be added to the reaction medium in a large range of concentrations. As non-limiting examples, one can cite as ozone concentration values ranging from 0.8 molar equivalents to 3 molar equivalents, relative to the amount of the compound of formula (II). Preferably, the ozone concentration will be comprised between 1.0 molar equivalents to 1.2 molar equivalents. It goes without saying that the optimum concentration of ozone will depend, as the person skilled in the art knows, on the nature of the compound of formula (II), the desired conversion, as well as the desired time of reaction.

The reducing agent can be added to the reaction medium in a large range of concentrations. As non-limiting examples, one can cite as reducing agent concentration values those ranging from 0.5 molar equivalents to 10 molar equivalents, relative to the amount of compound of formula (II). Preferably, the reducing agent concentration will be comprised between 0.8 molar equivalents to 10 molar molar equivalents. Even more preferably, the reducing agent concentration will be comprised between 2.0 molar equivalents to 5 molar equivalents. It goes without saying that the optimum concentration of reducing agent will depend, as the person skilled in the art knows, on the nature of the latter, on the nature of the compound of formula (II), the desired conversion, as well as the desired time of reaction.

The conditions used to convert the intermediate of formula (III) to the compound of formula (I) will depend on the nature of the X′ group.

According to a particular embodiment of the invention, when X′ is a CH₂OR² group, the conversion of the intermediate of formula (III) to the compound of formula (I) comprises a retro aldol reaction. When R² is not a hydrogen atom, a deprotection is carried out prior to the retro aldol. The deprotection step will depend on the nature of the R² group. The person skilled in the art will be able to select the best conditions. For example, when R² is a C(═O)R^{a} group wherein R^{a} is a hydrogen atom or a C₁₋₆ alkyl group, the deprotection to form a compound of formula (III) wherein X′ is a CH₂OH group may be carried out under normal condition known by the person skilled in the art, e.g. in the presence of water and an acid, preferably an acid having a pH in the range comprised between 1 and 2 such as H₂SO₄, pTsOH, oxalic acid or phosphoric acid; or in the presence of an enzyme such as lipase.

According to a particular embodiment of the invention, the retro aldol reaction is a thermal retro aldol reaction.

The temperature at which the retro-aldol reaction can be carried out is comprised between 450° C. and 550° C., particularly, in the range of between 490° C. and 550° C., particularly, in the range of between 490° C. and 550° C., particularly, in the range of between 49° and 530° C., even more particularly, in the range of between 49° and 510° C. Of course, a person skilled in the art is able to select the preferred temperature as a function of the melting and boiling point of the starting and final products as well as the desired time of reaction or conversion.

The retro-aldol reaction can be carried out in the presence or absence of a solvent. When a solvent is required or used for practical reasons, then any solvent current in such reaction type can be used for the purposes of the invention. In particular, the retro aldol reaction is carried out in a presence of a solvent having a boiling point equal or greater than 65° C., even greater than 75° C., even greater than 80° C., even greater than 110° C., even greater than 130° C. Non-limiting examples of suitable solvents include alcoholic solvent such as methanol, ethanol, 1-propanol, isopropanol, 1-hexanol, 1-octanol, 1-butanol, 1-pentanol, 4-methylpentan-2-ol, 2-methyl-1-pentanol, 1-heptanol, 2-octylalcohol, cyclohexanol or mixtures thereof, C₆₋₁₂ aromatic solvents such as toluene, xylene, or mixtures thereof, hydrocarbon solvents such as n-heptane, n-decane, n-dodecane, n-nonane, cyclohexane or a mixture thereof, ethereal solvents such diisobutyl ether or di n-butylether or mixtures thereof, solvent comprising a ketone functional group such as 4-methyl-2-pentanone MIBK, 2-octanone, cyclohexanonone, 2-heptanone, 6-methyl-2-heptanone, 6-methyl-5-hepten-2-one, isophorone or a mixture thereof, solvent comprising a aldehyde functional group such as hexanal, octanal or a mixture thereof. The choice of the solvent is function of the nature of the substrate and the person skilled in the art is well able to select the solvent most suitable in each case to optimize the reaction.

The retro aldol reaction may be carried out under batch or continuous conditions.

Unexpectedly, the thermal retro aldol reaction allows limiting side product formation. The conditions reported in the prior art for such conversions, such as non-industrially applicable Jones Oxidation, does not allow obtaining compound of formula (I) in high yield. According to a particular embodiment of the invention's process, at most 20% of undesired eliminated product of formula (VII)

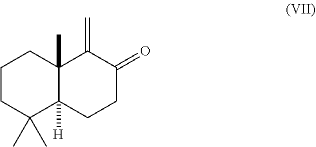

is formed.

According to a particular embodiment of the invention, when X′ is a COOH or a C(═O)OR¹ group wherein R¹ has the same meaning as defined above, the conversion of the intermediate of formula (III) to the compound of formula (I) comprises a decarboxylation reaction. The decarboxylation reaction may be carried out under normal condition known by the person skilled in the art, i.e. in the presence of a base such as an alkali metal hydroxide, e.g. NaOH or KOH or in the presence of an acid such as Bronsted acid or Lewis acid. Particularly, the decarboxylation is performed in the presence of a base when X′ is a C(═O)OR¹ group wherein R¹ has the same meaning as defined above. The decarboxylation is well known and has been largely reported in the prior art. So, the person skilled in the art will be able to set up the best conditions in order to convert compound of formula (III), wherein X′ is a COOH or a C(═O)OR¹ group wherein R¹ has the same meaning as defined above, into compound of formula (I). As non-limiting example, the decarboxylation reaction may be performed under the conditions reported in WO2012069647. Specific and non-limiting examples of Bronsted acid or Lewis acid may be selected from the group consisting of diluted sulfuric acid, para toluene sulfonic acid, methane sulfonic acid, camphor sulfonic acid, Triflic acid, methane disulfonic acid, methane trisulfonic acid, 2,4 dinitrobenzene sulfonic acid, diluted HCl and Al₂O₃.

The acid or base can be added into the reaction medium of the invention's process in a large range of concentrations. As non-limiting examples, one can cite as acid or base concentration values those ranging from about 1 to about 20 mol %, relative to the amount of the of substrate, preferably from 5 to about 10 mol %, relative to the amount of the of substrate The optimum concentration of the acid or base will depend, as the person skilled in the art knows, on the nature of the latter, on the nature of the substrate, on the reaction temperature as well as on the desired time of reaction.

According to any one of the invention's embodiments, the decarboxylation reaction may be carried out at a temperature comprised between 20° C. and 80° C. In particular, the temperature is in the range between 20° C. and 60° C. Of course, a person skilled in the art is also able to select the preferred temperature as a function of the melting and boiling point of the starting and final products as well as the desired time of reaction or conversion.

The decarboxylation reaction can be carried out in the presence or absence of a solvent. When a solvent is required or used for practical reasons, then any solvent current in such reaction type can be used for the purposes of the invention. Non-limiting examples include C₆₋₁₂ aromatic solvents such as toluene, xylene, 1,3-diisopropylbenzene, cumene or pseudocumene, or mixtures thereof, alcoholic solvent such as methanol, ethanol, or mixtures thereof, hydrocarbon solvents such as cyclohexane or heptane, ethyl acetate or ethereal solvents such as dioxane, methyl tetrahydrofuran, tetrahydrofuran or mixtures thereof. The choice of the solvent is function of the nature of the substrate and/or base or acid and the person skilled in the art is well able to select the solvent most suitable in each case to optimize the reaction.

According to a particular embodiment of the invention, when X′ is a CHO group, the conversion of the intermediate of formula (III) to the compound of formula (I) comprises a decarbonylation reaction or, alternatively, a oxidation reaction followed by a decarboxylation reaction. The decarbonylation reaction may be carried out under normal condition known by the person skilled in the art, i.e. in the presence of an alkali metal hydroxide, e.g. NaOH or KOH; and a RCOOM wherein R is a C1-8 alkyl group and M is a alkali metal such as KOAc. The oxidation reaction may be carried out under normal condition known by the person skilled in the art such as under Jones conditions. The decarboxylation reaction may be carried out under conditions reported above.

According to any embodiment of the invention, the compound of formula (I) may be converted into a compound of formula

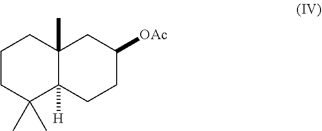 [SMILES: CC(=O)O[C@H]1CC[C@H]2C(C)(C)CCC[C@]2(C)C1]

wherein the bold and hatched lines indicate a relative or absolute configuration. The conversion of compound of formula (I) into compound of formula (IV) has been largely reported in the prior arts, such as in Journal of Chemical Research, Synopses (1998), (1), 36-37).

The compound of formula (II) when X is a vinyl group is a novel compound and present a number of advantages as explained above and shown in the Examples. Therefore, another object of the present invention is a compound of formula

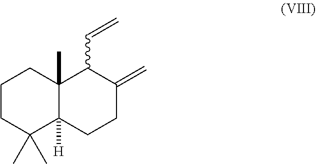 [SMILES: C=CC1C(=C)CC[C@H]2C(C)(C)CCC[C@]12C]

wherein the bold and hatched lines indicate a relative or absolute configuration.

The compound of formula (III) when X′ is a COOH group is a novel compound and present a number of advantages as explained above and shown in the Examples. Therefore, another object of the present invention is a compound of formula

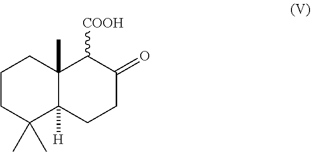 [SMILES: CC1(C)CCC[C@]2(C)C(C(=O)O)C(=O)CC[C@@H]12]

wherein the bold and hatched lines indicate a relative or absolute configuration.

The ozonolyis of compounds of formula (II) provides ozonide intermediates which are novel compounds and present a number of advantages as explained above and shown in the Examples. So another object of the present invention is compound of formula

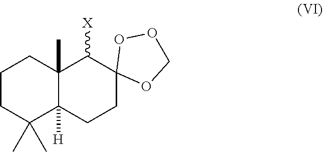

in the form of any one of its stereoisomers or a mixture thereof, and wherein the bold and hatched lines indicate a relative or absolute configuration; X represents a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═O)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group or one R^{c} and R^{d}, when taken together, form a C₄₋₅ oxacycloalkyl group.

An embodiment of the invention is wherein the compound of formula (II) used in the presently claimed process is obtained by contacting farnesyl pyrophosphate with at least one enzyme.

In some embodiments of the presently claimed process enzymes are used to prepare compounds of formula (II). For example, compound of formula (II) wherein X is a CH₂OR² group wherein R² represents a hydrogen atom or a C(═O)Me group may be produced using a Haloacid dehalogenase-like (HAD-like) hydrolase superfamily as reported in WO2018220113 or WO2019229064 and then followed, when X is a CH₂OR² group wherein R² is a C(═O)Me group, by an acetyl transferase as disclosed in WO2020078871.

In some embodiments of the presently claimed process enzymes are used to prepare compounds of formula (II).

The process to prepare compounds of formula (II) can be carried out in vitro as well as in vivo, as will be explained in details further on.

When the process is carried out in vitro, the enzyme to use can be obtained by extraction from any organism expressing it, using standard enzyme extraction technologies. If the host organism is an unicellular organism or cell the enzyme may simply be collected from the culture medium, for example by centrifugation, optionally followed by washing steps and re-suspension in suitable buffer solutions. If the organism or cell accumulates the enzyme within its cells, the enzyme may be obtained by disruption or lysis of the cells and further extraction of the enzyme from the cell lysate.

For the in vitro method the enzyme can be provided in isolated form or as part of a protein extract and is suspended in a buffer solution at optimal pH. If adequate, salts, DTT, NADPH, NADH, FAD, FMN and other kinds of enzymatic co-factors, may be added in order to optimize enzyme activity. The precursor compound is then added to the reaction mixture and incubated at optimal temperature, for example between 15 and 40° C., preferably between 25 and 35° C., more preferably at 30° C. After incubation, the compounds of formula (II) produced may be isolated from the incubated solution by standard isolation procedures, such as solvent extraction and distillation, optionally after removal of enzymes from the solution.

According to another preferred embodiment, the process to prepare compounds of formula (II) is carried out in vivo. In this case, the process comprises cultivating a non-human host organism or cell transformed to express the enzyme in the presence of a starting compound to be converted into the compounds of formula (II) under conditions conducive to the enzymatic reaction.

In an embodiment, in the case where a host cell is used or when the host organism is a microorganism, the compound to be converted can be added to the culture medium of said cell or microorganism. The starting compound will permeate through the membrane of the cell or microorganism, thus being available for reaction with the enzyme expressed by said host cell or microorganism.

Carrying out the method in vivo is particularly advantageous since it is possible to carry out the method without previously isolating the enzyme. The reaction occurs directly within the organism or cell transformed to express the enzyme.

To carry out the invention in vivo, the host organism or cell is cultivated under conditions conducive to the production of the compounds of formula (II). Such conditions are any conditions leading to growth of the host organism or cell. Preferably, such conditions are designed for optimal growth of the host organism or cell. If the host is a unicellular organism, conditions conducive to the production of the compounds of formula (II) may comprise addition of suitable cofactors to the culture medium of the host. In addition, a culture medium may be selected, so as to maximize synthesis.

Optimal culture conditions are known to the person skilled in the art and are not specific to the present invention.

In a more preferred embodiment the organism used to carry out the method of the invention in vivo is a microorganism. Any microorganism can be used but according to an even more preferred embodiment said microorganism is a bacteria or fungus. Preferably said fungus is yeast. Most preferably, said bacteria is E. coli and said yeast is Saccharomyces cerevisiae.

Another object of the present invention is the use of compound of formula

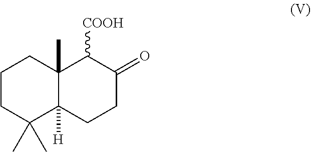 [SMILES: CC1(C)CCC[C@]2(C)C(C(=O)O)C(=O)CC[C@@H]12]

wherein the bold and hatched lines indicate a relative or absolute configuration; in the preparation of compound of formula (I) and (IV).

Another object of the present invention is the use of compound of formula

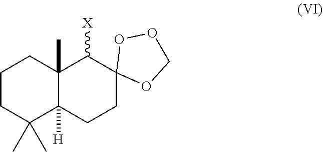

in the form of any one of its stereoisomers or a mixture thereof, and wherein the bold and hatched lines indicate a relative or absolute configuration; X represents a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═O)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group or one R^{c} and R^{d}, when taken together, form a C₄₋₅ oxacycloalkyl group;
in the preparation of compound of formula (I) and (IV).

Another object of the present invention is the use of compound of formula

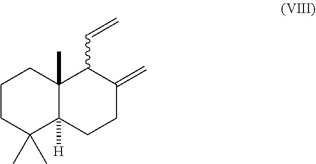 [SMILES: C=CC1C(=C)CC[C@H]2C(C)(C)CCC[C@]12C]

wherein the bold and hatched lines indicate a relative or absolute configuration; in the preparation of compound of formula (I) and (IV).

Typical manners to execute the invention's process are reported herein below in the examples.

### EXAMPLES

The invention will now be described in further detail by way of the following examples, wherein the abbreviations have the usual meaning in the art, the temperatures are indicated in degrees centigrade (° C.). The preparation of precatalysts and ligands solutions were carried out under an inert atmosphere (Argon) using standard Schlenk techniques. The solvents were dried by conventional procedures and distilled under an argon atmosphere. NMR spectra were recorded at 20° C. on Bruker AV 300, AV 400, or AV 500 MHz spectrometers. Chemical shifts are reported in ppm relative to solvent signals (chloroform, δ^{H}=7.26 ppm, δ_{C}=77.0 ppm). The signal assignment was ensured by recording ¹H,¹H-COSY, -NOESY, ¹³C,¹H-HSQC and -HMBC experiments. Gas chromatography was performed on an Agilent 7890 A Series equipped with a HP5 column (30 m×0.25 mm ID, 0.25 μm film) and tetradecane was used as internal standard.

### Example 1

### Preparation of ((1S,4aS,8aS)-5,5,8a-trimethyl-2-methylenedecahydronaphthalen-1-yl)methanol from ((1S,4aS,8aS)-5,5,8a-trimethyl-2-methylenedecahydronaphthalen-1-yl)methyl acetate

36.0 g (136.2 mmol) ((1S,4aS,8aS)-5,5,8a-trimethyl-2-methylenedecahydronaphthalen-1-yl)methyl acetate were dissolved under stirring in 480 mL MeOH in a 1 L flask in the presence of 38.3 g (578.7 mmol, 4.3 eq) KOH. After 2 hours a full conversion of starting material was observed. The solvent was partly evaporated under reduced pressure (45° C., 15 mbar), and 1.5 L diethyl ether and 500 mL water were added.
The aqueous phase was separated and the organic phase was washed twice with 500 mL water and once with with 500 mL of a saturated aqueous NaCl solution. The organic phase was dried over Na₂SO₄ and the solvent was evaporated under reduced pressure (45° C., 500-3 mbar). 100 mL pentane were added and the solvent was evaporated under reduced pressure (45° C., 500-3 mbar). A white solid (29.7 g ((1S,4aS,8aS)-5,5,8a-trimethyl-2-methylenedecahydronaphthalen-1-yl)methanol, 133.7 mmol, 98% yield) was obtained. ¹H and ¹³C NMR spectral data are identical to those reported in M. Göhl, K. Seifert, Eur. J. Org. Chem. 2014, 6975-6982.

### Example 2

### Preparation of (1S,4aS,8aS)-5,5,8a-trimethyl-2-methylenedecahydronaphthalene-1-carbaldehyde from ((1S,4aS,8aS)-5,5,8a-trimethyl-2-methylenedecahydronaphthalen-1-yl)methanol

15.61 g (70.2 mmol) of ((1S,4aS,8aS)-5,5,8a-trimethyl-2-methylenedecahydronaphthalen-1-yl)methanol (0.523 g (7.02 mmol) KCl, 2.84 g (7.02 mmol) Fe(NO₃·9H₂O and 1.097 g (7.02 mmol) TEMPO were dissolved in 150 mL toluene. Air was bubbled into the solution and the mixture was stirred 25.5 hours. A further quantity of catalyst was added (0.523 g (7.02 mmol) KCl, 2.84 g (7.02 mmol) Fe(NO₃)₃·9H₂O and 1.097 g (7.02 mmol) TEMPO), air was bubbled into the solution and the mixture was stirred for 21 hours. After the addition of 50 mL water and 75 mL Et₂O the mixture was stirred 10 min. The organic phase was separated and washed with 50 mL water and 50 mL of a saturated aqueous NaCl solution. After drying over Na₂SO₄ and filtration the solvent was evaporated under reduced pressure (18.73 g crude). The product was purified by column chromatography (80 g SiO₂, Et₂O/Cyclohexane 1/9). 13.7 g (1S,4aS,8aS)-5,5,8a-trimethyl-2-methylenedecahydronaphthalene-1-carbaldehyde of an orange oil were obtained (98.8% GC purity, 88% yield). Only 0.3 g (1.8% yield) of (1S,4aS,8aS)-5,5,8a-trimethyl-2-methylenedecahydronaphthalene-1-carboxylic acid were isolated. ¹H and ¹³C NMR spectral data are identical to those reported in Hua, D. H.; Huang, X.; Chen, Y.; Battina, S. K.; Tamura, M.; Noh, S. K.; Koo, S. I.; Namatame, I.; Tomada, H.; Perchellet, E. M.; Perchellet, J.-P. J. Org. Chem. 2004, 69, 6065-6078.

### Example 3

### Preparation of (1S,4aS,8aS)-5,5,8a-trimethyl-2-methylenedecahydronaphthalene-1-carboxylic acid from (1S,4aS,8aS)-5,5,8a-trimethyl-2-methylenedecahydronaphthalene-1-carbaldehyde

20.0 g (69.7 mmol) (1S,4aS,8aS)-5,5,8a-trimethyl-2-methylenedecahydronaphthalene-1-carbaldehyde, 13.08 g (218 mmol) AcOH, 28.5 g (386 mmol) isoamylene were added to 50 mL EtOAc. A mixture of 12.31 g (80% purity, 109 mmol) of NaClO₂ in 60 mL water was introduced slowly under stirring, maintaining the temperature between 25-38° C. At the end of the addition the mixture was heated 30 min at 40° C. (oilbath). After cooling down to room temperature 120 mL of a 10% aqueous Na₂S₂O₃ solution was added and stirring was continued for 15 minutes. Then a 5% aqueous NaOH solution was added until a slightly basic pH was measured. After 10 min of stirring a standard acid/base extraction (two extractions with Et₂O) was done. After evaporation of the solvent under reduced pressure one obtains 19.9 g as a crystalline solid. The product was purified by column chromatography (300 g SiO₂, AcOEt/Cyclohexane 2/8) and 17.4 g of (1S,4aS,8aS)-5,5,8a-trimethyl-2-methylenedecahydronaphthalene-1-carboxylic acid) was isolated as a white solid (99.1% purity, 80% yield).

¹H-NMR and ¹³C-NMR analysis results in CDCl₃ were in accordance with data from literature (T. Laube, J. Schroder, R. Stehle, K. Seifert, Tetrahedron 2002, 58, 4299-4309). ¹³C NMR (150 MHz, CDCl₃): δ 14.1, 18.9, 21.8, 23.2, 33.4, 33.5, 36.3, 39.1, 39.2, 42.0, 54.5, 63.0, 108.6, 143.2, 177.9.

(1S,4aS,8aS)-5,5,8a-trimethyl-2-methylenedecahydronaphthalene-1-carboxylic acid can also prepared from ((1S,4aS,8aS)-5,5,8a-trimethyl-2-methylenedecahydronaphthalen-1-yl)methanol using a Jones Oxidation (72% yield).

### Example 4

### Preparation of methyl (1S,4aS,8aS)-5,5,8a-trimethyl-2-methylenedecahydronaphthalene-1-carboxylate from (1S,4aS,8aS)-5,5,8a-trimethyl-2-methylenedecahydronaphthalene-1-carboxylic acid

2 g (8.46 mmol) of (1S,4aS,8aS)-5,5,8a-trimethyl-2-methylenedecahydronaphthalene-1-carboxylic acid were heated to reflux under stirring in the presence of 18 g (200 mmol) of dimethylcarbonate (92° C. boiling point of dimethylcarbonate) and 1.288 g (8.46 mmol) DBU. After 9 hours a complete conversion was achieved and the excess of dimethylcarbonate was evaporated under reduced pressure. 50 mL AcOEt were added and the solution was washed twice with a 5% aqueous HCl solution and once with a saturated aqueous NaCl solution. The organic phase was dried over Na₂SO₄ and the solvent was evaporated under reduced pressure (2.08 g methyl (1S,4aS,8aS)-5,5,8a-trimethyl-2-methylenedecahydronaphthalene-1-carboxylate, 98.5% purity, 97% yield). No further purification was necessary.

¹H-NMR and ¹³C-NMR analysis results in CDCl₃ were in accordance with data from literature (T. Laube, J. Schroder, R. Stehle, K. Seifert, Tetrahedron 2002, 58, 4299-4309). ¹³C NMR (150 MHz, CDCl₃): δ 14.2, 18.9, 21.7, 23.3, 33.4, 33.5, 36.3, 39.1, 39.3, 42.1, 50.9, 54.6, 63.1, 108.4, 143.8, 172.1.

Methyl (1S,4aS,8aS)-5,5,8a-trimethyl-2-methylenedecahydronaphthalene-1-carboxylate can also be prepared from methyl (1S,4aS,8aS)-5,5,8a-trimethyl-2-methylenedecahydronaphthalene-1-carboxylate in the presence of MeI and K₂CO₃ in acetone (4 h, 40° C., 91% yield).

### Example 5

### Preparation of (1S,4aS,8aS)-1-(hydroxymethyl)-5,5,8a-trimethyloctahydronaphthalen-2(1H)-one from ((1S,4aS,8aS)-5,5,8a-trimethyl-2-methylenedecahydronaphthalen-1-yl)methanol

13.0 g (98.1% purity, 57.4 mmol) ((1S,4aS,8aS)-5,5,8a-trimethyl-2-methylenedecahydronaphthalen-1-yl)methanol were dissolved in 600 mL MeOH in an ozonolysis reactor. The mixture was cooled to 0° C. and ozone was bubbled inside under stirring until a complete conversion of the starting material was observed by GC (KI test positive). After that oxygen (10 min) and nitrogen (10 min) were bubbled into the mixture. 7.13 g (114.7 mmol, 2 eq) Me₂S were added at 0° C. under stirring. The cooling was removed after 1.5 h and the mixture was stirred overnight at room temperature. As the KI test was slightly positive, 15.0 g (57.4 mmol, 1 eq) PPh₃ was added. After 15 min the KI test was negative and the GC showed 96.7% of the product. The mixture was filtered and the solvent was evaporated under reduced pressure (Rotavap in the fumehood, 45° C., 50-5 mbar). Some pentane and dichloromethane were added and evaporated under reduced pressure (crude 32.9 g). The crude (mixed with small amounts of dichloromethane) was purified by flash chromatography (2×330 g SiO₂, eluent cyclohexane 7/EtOAc 3). 12.92 g (98.6% purity, 56.8 mmol, 99.0% yield) of (1S,4aS,8aS)-1-(hydroxymethyl)-5,5,8a-trimethyloctahydronaphthalen-2(1H)-one were obtained as a white solid.

¹H-NMR and ¹³C-NMR analysis results in CDCl₃ were in accordance with data from literature (Furuichi, N.; Hata, T.; Soetjipto, H.; Kato, M.; Katsumura, S. Tetrahedron 2001, 57, 8425-8442).

¹H-NMR (500.15 MHz, CDCl₃): 0.81 (s, 3H), 0.87 (s, 3H), 0.98 (s, 3H), 1.23-1.34 (m, 2H), 1.44-1.56 (m, 4H), 1.64-1.73 (m, 2H), 2.02-2.08 (m, 1H), 2.29-2.36 (m, 2H), 2.47 (ddd, 1H J=14.4 Hz, J=5.0 Hz, J=1.9 Hz), 3.60 (brd, 1H, J=9.8 Hz) 3.96 (dd, 1H, J=11.2 Hz, J=9.5 Hz).

¹³C NMR (150 MHz, CDCl₃): δ 15.9, 18.8, 21.8, 23.3, 33.6, 33.6, 39.1, 41.1, 41.7, 42.1, 53.5, 57.8, 65.4, 214.8.

The reduction of the Ozonide can be also done with the mono Na salt of 3,3′-Thiodipropionic acid (2 eq, 99% yield).

The reduction of the Ozonide can be also done with the thiodiglycol (2 eq, 99% yield).

The reduction of the Ozonide can be also done with the thiourea (0.5 eq, 95% yield).

The same yield could be also obtained in AcOH.

Depending on the reducing reagent and on the pH before the solvent evaporation up to 6% of (1S,4aS,8aS)-1-((hydroxymethoxy)methyl)-5,5,8a-trimethyloctahydronaphthalen-2(1H)-one could be identified by NMR-spectroscopy (or GC, after silylation). The content of (1S,4aS,8aS)-1-((hydroxymethoxy)methyl)-5,5,8a-trimethyloctahydronaphthalen-2(1H)-one in (1S,4aS,8aS)-1-(hydroxymethyl)-5,5,8a-trimethyloctahydronaphthalen-2(1H)-one did not influence the selectivity and yield of the thermal retro aldol reaction to (4aS,8aR)-5,5,8a-trimethyloctahydronaphthalen-2(1H)-one. (1S,4aS,8aS)-1-((hydroxymethoxy)methyl)-5,5,8a-trimethyloctahydronaphthalen-2(1H)-one

¹³C NMR (150 MHz, CDCl₃):

δ 15.5, 18.9, 21.7, 23.9, 33.5, 33.7, 39.3, 41.8, 41.9, 42.3, 54.0, 62.0, 64.0, 90.3, 211.4. NMR signal for the the silylated (1S,4aS,8aS)-1-((hydroxymethoxy)methyl)-5,5,8a-trimethyloctahydronaphthalen-2(1H)-one: (1S,4aS,8aS)-5,5,8a-trimethyl-1-((((trimethylsilyl)oxy)methoxy)methyl)octahydronaphthalen-2(1H)-one

¹³C NMR (125 MHz, CDCl₃): 3-0.2, 15.4, 18.9, 21.7, 24.0, 33.5, 33.7, 39.3, 41.9, 42.0, 42.4, 54.1, 62.0, 63.9, 90.3, 210.5

### Example 6

### Preparation of (1S,4aS,8aS)-1-(hydroxymethyl)-5,5,8a-trimethyloctahydronaphthalen-2(1H)-one from ((1S,4aS,8aS)-5,5,8a-trimethyl-2-methylenedecahydronaphthalen-1-yl)methanol with isolation of intermediate ozonide

1.00 g ((1S,4aS,8aS)-5,5,8a-trimethyl-2-methylenedecahydronaphthalen-1-yl)methanol (4.50 mmol) were dissolved in the Ozonolysis flask in 90 mL EtOAc. The mixture was cooled to 0° C. and Oxygen was bubbled into the mixture under stirring for 10 min. Then Ozone was bubbled into the solution for 7 min at 0° C. Finally Oxygen and Nitrogen were bubbled into the mixture for 10 min at 0° C. The intermediate (((1S,4aS,8aS)-5,5,8a-trimethyloctahydro-1H-spiro[naphthalene-2,3′-[1,2,4]trioxolan]-1-yl)methanol) was analysed by NMR-spectroscopy.

¹H-NMR (500.15 MHz) characteristic signals: 0.82 (s, 3H), 0.83 (s, 3H), 0.89 (s, 3H), 1.81-1.87 (m, 1H), 1.91 (dd, 1H, J=6.9 Hz, J=2.1 Hz) 3.66 (dd, 1H, J=11.8 Hz, J=2.2 Hz), 3.92 (dd, 1H, J=11.7 Hz, J=16.6 Hz), 4.95 (s, 1H), 5.28 (s, 1H).

¹³C NMR (125 MHz, CDCl₃): 315.6, 18.6, 19.7, 21.6, 33.2, 33.6, 36.4, 38.6, 39.1, 41.7, 54.6, 58.2, 59.1, 93.0, 111.7.

To this intermediate was added 2.36 g (8.99 mmol 2 eq) PPh₃ and the mixture was warmed up slowly to room temperature (2 hours). After stirring overnight and washing with a saturated aqueous NaCl solution, the mixture was dried over Na₂SO₄ and the solvent was evaporated under reduced pressure (Rotavap in the fume hood). The crude was purified by flash chromatography (100 g SiO₂, eluent cyclohexane 7/EtOAc 3). 0.969 g (4.32 mmol, 96% yield) of (1S,4aS,8aS)-1-(hydroxymethyl)-5,5,8a-trimethyloctahydronaphthalen-2(1H)-one were obtained as a white solid.

The same yield (>95% yield) could be also obtained in CH₂Cl₂.

### Example 7

### Preparation of (4aS,8aR)-5,5,8a-trimethyloctahydronaphthalen-2(1H)-one from (1S,4aS,8aS)-1-(hydroxymethyl)-5,5,8a-trimethyloctahydronaphthalen-2(1H)-one

2 g (97.8% purity, 8.72 mmol) (1S,4aS,8aS)-1-(hydroxymethyl)-5,5,8a-trimethyloctahydronaphthalen-2(1H)-one were dissolved in 26 mL 1-octanol. The mixture was added slowly (26 mL/h) from the top (under a N₂ flow) to a heated pyrolysis column (23×2 cm, pyrolysis oven at 500° C.), which was filled with 56 g glass beads and was connected to a water cooled condenser and a 100 mL flask under the pyrolysis column. When the addition was finished (1 hour), the oven was cooled down. When 50° C. oven temperature were reached, the column was washed twice with 5 mL cyclohexane. The cyclohexane was evaporated under reduced pressure and the 1-octanol was distilled off under reduced pressure (Vigreux column). The crude (GC 89%) was purified by flash chromatography (120 g SiO₂, eluent cyclohexane 8/EtOAc 2). 1376 mg (7.08 mmol, 81.2% yield) (4aS,8aR)-5,5,8a-trimethyloctahydronaphthalen-2(1H)-one were obtainedas a white solid.

¹H-NMR and ¹³C-NMR analysis results in CDCl₃ were in accordance with data from literature (A. Gautier, C. Vial, C. Morel, M. Lander, F. Naf, Helv. Chim. Acta, 1987, 70, 2039 and I. Jabin, G. Revial, K. Melloul, M. Pfau, Tetrahedron: Asymmetry 1997, 8, 1101-1109)

¹³C NMR (90 MHz, CDCl₃): 318.8, 19.4, 21.4, 23.1, 33.2, 33.2, 38.4, 42.0, 42.0, 42.4, 52.2, 59.6, 211.6.

### Example 8

### Preparation of (4aS,8aR)-5,5,8a-trimethyloctahydronaphthalen-2(1H)-one and (1R,4aS,8aS)-5,5,8a-trimethyl-2-oxodecahydronaphthalene-1-carboxylic acid from (1S,4aS,8aS)-5,5,8a-trimethyl-2-methylenedecahydronaphthalene-1-carbaldehyde

3.92 g (98.8% purity, 17.81 mmol) (1S,4aS,8aS)-5,5,8a-trimethyl-2-methylenedecahydronaphthalene-1-carbaldehyde were dissolved in 120 mL EtOAc in an ozonolysis reactor. The mixture was cooled to 0° C. and ozone was bubbled inside under stirring until a complete conversion of the starting material was observed by GC (30 min, KI test positive). After that oxygen (10 min) and nitrogen (10 min) were bubbled into the mixture. 10.5 g (40.1 mmol, 2.25 eq) PPh₃ were added. The cooling was removed after 1.5 h and the mixture was stirred overnight at room temperature (KI test was negative). The mixture was washed twice with a 2.5% aqueous NaOH solution. The organic phase was washed with a saturated aqueous NaCl solution, was dried over Na₂SO₄ and the solvent was evaporated under reduced pressure (Rotavap in the fumehood). The crude was purified by flash chromatography (120 g SiO₂, eluent cyclohexane 8/EtOAc 2). 368 mg (1.89 mmol, 11% yield) (4aS,8aR)-5,5,8a-trimethyloctahydronaphthalen-2(1H)-one were obtained as a white solid.

An 5% aqueous HCl solution was added slowly to the combined aqueous phases (2.5% aqueous NaOH solution) until pH 4 was reached. The aqueous phase was extracted twice with 100 mL EtOAc. The combined organic phases were washed with a saturated aqueous NaCl solution and were dried over Na₂SO₄ and the solvent was evaporated under reduced pressure (4.70 g). The crude was purified by flash chromatography (220 g SiO₂, eluent cyclohexane 8/EtOAc 2 to cyclohexane 6/EtOAc 4). 2 626 mg (11.02 mmol, 62% yield) (1R,4aS,8aS)-5,5,8a-trimethyl-2-oxodecahydronaphthalene-1-carboxylic acid were obtained as a white solid. (1R,4aS,8aS)-5,5,8a-trimethyl-2-oxodecahydronaphthalene-1-carboxylic acid:

¹H-NMR (500.15 MHz, CDCl₃) characteristic signals: δ0.88 (s, 3H), 0.98 (s, 3H), 1.05 (s, 3H), 1.23-1.30 (m, 1H), 1.46-1.61 (m, 5H), 1.72-1.82 (m, 2H), 2.09-2.17 (m, 2H), 2.43 (ddd, 1H, J=13.5 Hz, J=13.5 Hz, J=7.5 Hz), 2.54 (ddd, 1H, J=13.9 Hz, J=5.0 Hz, J=1.8 Hz), 3.27 (s, 1H), 12.56 (COOH).

¹³C NMR (150 MHz, CDCl₃): δ 15.1, 18.7, 21.6, 23.8, 33.5, 33.8, 39.3, 41.6, 41.7, 43.6, 53.5, 68.8, 171.7, 208.8.

(1R,4aS,8aS)-5,5,8a-trimethyl-2-oxodecahydronaphthalene-1-carboxylic acid can be transformed to (4aS,8aR)-5,5,8a-trimethyloctahydronaphthalen-2(1H)-one in the presence of diluted sulfuric acid (and heating). A quantitative yield was obtained.

### Example 9

### Preparation of (4aS,8aR)-5,5,8a-trimethyloctahydronaphthalen-2(1H)-one and (1R,4aS,8aS)-5,5,8a-trimethyl-2-oxodecahydronaphthalene-1-carboxylic acid from (1S,4aS,8aS)-5,5,8a-trimethyl-2-methylenedecahydronaphthalene-1-carboxylic acid

0.617 g (2.613 mmol) (1S,4aS,8aS)-5,5,8a-trimethyl-2-methylenedecahydronaphthalene-1-carboxylic acid were dissolved in 30 mL MeOH in an ozonolysis reactor. The mixture was cooled to 0° C. and ozone was bubbled inside under stirring until a complete conversion of the starting material was observed by GC (10 min, KI test positive). After that oxygen (10 min) and nitrogen (10 min) were bubbled into the mixture. 0.399 g (6.414 mmol) Me₂S were added at 0° C. under stirring. The cooling was removed after 1.5 h and the mixture was stirred overnight at room temperature (KI test was negative). The solvent was evaporated under reduced pressure (in the fume hood). The crude was dissolved in diethyl ether and was washed with a 5% aqueous HCl solution, with water (3 times), and with saturated aqueous NaCl solution. The aqueous phases were reextracted with diethyl ether and the combined organic phases were washed with a 5% aqueous NaOH solution. A precipitation was observed (not soluble in water, Na salt of (1R,4aS,8aS)-5,5,8a-trimethyl-2-oxodecahydronaphthalene-1-carboxylic acid). The suspension was extracted twice with diethyl ether. The organic phases were separated and the suspension was treated with a 5% aqueous sulfuric acid solution. A CO₂ formation was observed during stirring at room temperature. After 20 min the mixture was heated for 10 min at 50° C. After cooling down to room temperature 0.340 mg (1.75 mmol, 67% yield) of an insoluble white solid ((4aS,8aR)-5,5,8a-trimethyloctahydronaphthalen-2(1H)-one) could be separated from the aqueous phase.

### Example 10

### Preparation of methyl (1R,4aS,8aS)-5,5,8a-trimethyl-2-oxodecahydronaphthalene-1-carboxylate from methyl (1S,4aS,8aS)-5,5,8a-trimethyl-2-methylenedecahydronaphthalene-1-carboxylate

13.66 g (54.6 mmol) methyl (1S,4aS,8aS)-5,5,8a-trimethyl-2-methylenedecahydronaphthalene-1-carboxylate (54.27 mmol) were dissolved in the Ozonolysis flask in 350 mL MeOH. The mixture was cooled to 0° C. and under stirring Ozone was bubbled into the solution for three hours. Then Oxygen was bubbled in for 15 min at 0° C. Dimethylsulfide (8.53 g, 137 mmol) was added quickly and the mixture was stirred 1.5 hours under Nitrogen bubbling (into the solution) at 0° C. After that the reaction was stirred 18.5 hours at room temperature. After the KI control (which has to be negative) the solvent was evaporated at reduced pressure (40° C.) with a Rotavap in the fume hood. 13.34 g methyl (1R,4aS,8aS)-5,5,8a-trimethyl-2-oxodecahydronaphthalene-1-carboxylate (52.6 mmol, 96% yield) were obtained. The product was not further purified and was used for the next step.

¹H-NMR and ¹³C-NMR analysis results in CDCl₃ were in accordance with data from literature (Y. Tanada, K. Mori, Eur. J Org. Chem. 2003, 848-854).

### Example 11

### Preparation of (4aS,8aR)-5,5,8a-trimethyloctahydronaphthalen-2(1H)-one from methyl (1R,4aS,8aS)-5,5,8a-trimethyl-2-oxodecahydronaphthalene-1-carboxylate

13.34 g methyl (1R,4aS,8aS)-5,5,8a-trimethyl-2-oxodecahydronaphthalene-1-carboxylate (52.6 mmol, from the previous step) were added to a mixture of 39.5 g (705 mmol) KOH, 78 mL water and 78 mL EtOH (flask with reflux condenser). After heating at 120° C. (oil bath) for 26 hours the mixture was cooled down to room temperature. The reaction was added to ice water and was extracted three times with 500 mL Et₂O. The combined organic phases were washed with 500 mL water, with 500 mL of a saturated aqueous NaCl solution and the organic phase was dried over Na₂SO₄. After evaporation of the solvent at reduced pressure one obtains 11.02 g (GC 94.5% purity) of the crude product. The product was purified by column chromatography (330 g SiO₂, EtOAc/Cyclohexane 13/87->EtOAc/Cyclohexane 3/7). 8.66 g (44.58 mmol, 85% yield) (4aS,8aR)-5,5,8a-trimethyloctahydronaphthalen-2(1H)-one were obtained as a white solid.

The preparation of (−)-Polywood ((2S,4aS,8aR)-5,5,8a-trimethyldecahydronaphthalen-2-yl acetate) from (4aS,8aR)-5,5,8a-trimethyloctahydronaphthalen-2(1H)-one was described in the literature:

### Cortes, Manuel; Moreno, Luis; Lopez, Jose Journal of Chemical Research, Synopses (1998), (1), 36-37).

### Example 12

### Preparation of compounds of formula (II) starting from ((1S,4aS,8aS)-5,5,8a-trimethyl-2-methylenedecahydronaphthalen-1-yl)methanol

### a) Preparation of ((1S,4aS,8aS)-5,5,8a-trimethyl-2-methylenedecahydronaphthalen-1-yl)methyl formate

2 eq Ac₂O, 2 eq formic acid, 1 h, 50° C., toluene, 97% yield

¹H-NMR (500.15 MHz, CDCl₃): 0.77 (s, 3H), 0.82 (s, 3H), 0.89 (s, 3H), 1.14 (dd, 1H, J=12.6 Hz, J=2.7 Hz), 1.17-1.29 (m, 2H), 1.29-1.39 (m, 1H), 1.39-1.44 (m, 1H), 1.47-1.62 (m, 2H), 1.69-1.77 (m, 2H), 2.00-2.22 (m, 2H), 2.40-2.44 (m, 1H), 4.26 (dd, 1H, J=11.1 Hz, J=9.6 Hz), 4.49 (dd, 1H, J=11.3 Hz, J=3.4 Hz), 4.52-4.55 (m, 1H), 4.88-4.90 (m, 1H), 8.04 (s, 1H).

¹³C NMR (125 MHz, CDCl₃): δ 15.2, 19.1, 21.8, 23.9, 33.5, 33.6, 37.5, 39.0, 39.1, 41.9, 54.8, 55.0, 61.0, 107.2, 146.6, 161.3.

### b) Preparation of trimethyl(((1S,4aS,8aS)-5,5,8a-trimethyl-2-methylenedecahydronaphthalen-1-yl)methoxy)silane

3 eq TMSC1, 3 eq NEt₃, 83% yield

¹H-NMR (500.15 MHz, CDCl₃): 0.16 (s, 9H), 0.73 (s, 3H), 0.85 (s, 3H), 0.97 (s, 3H), 1.23-1.24 (m, 1H), 1.30-1.39 (m, 1H), 1.43-1.49 (m, 1H), 1.50-1.56 (m, 3H), 1.59-1.70 (m, 1H), 1.76-1.81 (m, 1H), 2.02-2.10 (m, 1H), 2.30-2.40 (m, 2H), 2.41-2.47 (m, 1H), 3.63 (dd, 1H, J=9.6 Hz, J=3.1 Hz), 3.87 (dd, 1H, J=9.6 Hz, J=7.6 Hz), 4.81 (d, 1H, J=5.3 Hz), 4.86 (d, 1H, J=5.3 Hz).

¹³C NMR (150 MHz, CDCl₃): 3-0.4, 15.2, 19.3, 21.8, 24.0, 33.5, 33.7, 37.9, 39.0, 39.2, 42.0, 55.2, 58.2, 59.0, 107.2, 147.6.

### c) Preparation of (4aS,5S,8aS)-5-((I-ethoxyethoxy)methyl)-1,1,4a-trimethyl-6-methylenedecahydronaphthalene

3 eq Ethylvinylether, 0.11 eq CF₃COOH, 30° C., 2.5 h 90% yield

¹H-NMR (500.15 MHz, CDCl₃): 0.73 (s, 3H), 0.81 (s, 3H), 0.87 (s, 3H), 1.08-1.15 (m, 1H), 1.16-1.28 (m, 1H), 1.20 (td, 3H, J=7.1 Hz, J=1.4 Hz), 1.28-1.38 (m, 2H), 1.30 (dd, 3H, J=5.3 Hz, J=3.2 Hz), 1.37-1.45 (m, 1H), 1.45-1.59 (m, 2H), 1.67-1.79 (m, 2H), 1.92-1.99 (m, 1H), 2.02-2.11 (m, 1H), 2.37-2.43 (m, 1H), 3.43-3.52 (m, 1.5H), 3.60-3.69 (m, 2H), 4.26 (dd, 0.5H, J=9.7 Hz, J=3.4 Hz), 4.59-4.61 (m, 0.5H), 4.66-4.68 (m, 0.5H), 4.65-4.72 (m, 1H), 4.85-4.87 (m, 0.5H), 4.87-4.89 (m, 0.5H).

¹³C NMR (125 MHz, CDCl₃): δ 15.3, 15.4, 19.3, 19.8, 21.8, 24.0, 33.5, 33.7, 37.8, 38.9, 39.2, 42.0, 55.2, 56.1, 60.5, 62.1, 99.9, 107.3, 147.7.

¹³C NMR (125 MHz, CDCl₃): δ 15.3, 15.3, 19.3, 19.6, 21.8, 24.0, 33.5, 33.7, 37.8, 38.8, 39.2, 42.0, 55.2, 56.1, 60.3, 61.0, 99.4, 107.2, 147.6.

### d) Preparation of (4aS,5S,8aS)-5-((benzyloxy)methyl)-1,1,4a-trimethyl-6-methylenedecahydronaphthalene 1.05 eq Benzyl chloride, 1.05 eq KOtBu, 43 h, rt, THF, 90% yield

¹H-NMR (500.15 MHz, CDCl₃): 0.71 (s, 3H), 0.80 (s, 3H), 0.87 (s, 3H), 1.12 (dd, 1H, J=12.5 Hz, J=2.6 Hz), 1.15-1.23 (m, 2H), 1.27-1.36 (m, 1H), 1.36-1.41 (m, 1H), 1.43-1.57 (m, 1H), 1.65-1.74 (m, 2H), 2.01-2.10 (m, 1H), 2.36-2.44 (m, 2H), 2.41-2.47 (m, 1H), 3.57 (dd, 1H, J=8.7 Hz, J=8.7 Hz), 3.65 (dd, 1H, J=9.5 Hz, J=3.5 Hz), 4.45-4.54 (m, 2H), 4.59-4.61 (m, 1H), 4.86-4.88 (m, 1H), 7.26-7.40 (m, 5H).

¹³C NMR (150 MHz, CDCl₃): δ 15.3, 19.2, 21.8, 24.0, 33.5, 33.7, 37.8, 38.9, 39.2, 42.0, 55.1, 56.0, 67.0, 73.2, 107.3, 127.5, 127.8, 128.3, 138.6, 147.6.

### Example 13

### Preparation of compounds of formula (III) by oxidative cleavage of compounds of formula (II)

The starting material prepared in Example 12 (38.94 mmol) was dissolved in 100 mL solvent in an ozonolysis reactor. The mixture was cooled to 0° C. and ozone was bubbled inside under stirring until a complete conversion of the starting material was observed by GC (50 min, KI test positive). After that oxygen (5 min) and nitrogen (5 min) were bubbled into the mixture. 20.43 g (77.88 mmol, 2 eq) PPh₃ were added. The cooling was removed after 1.5 h and the mixture was stirred 3 h at room temperature (KI test was negative). In the case of MeOH as the solvent the solvent was evaporated under reduced pressure (Rotavap in the fumehood) and 100 mL EtOAc were added. The organic phase was washed with a saturated aqueous NaHCO₃ solution (50 mL) and water (50 mL). The solvent was evaporated under reduced pressure (Rotavap in the fumehood). The crude was purified by flash chromatography (Triphenylphosphine oxide can be eliminated by crystallizations in pentane (50 mL) before the flash chromatography).

### a) ((1S,4aS,8aS)-5,5,8a-trimethyl-2-oxodecahydronaphthalen-1-yl)methyl formate (Ozonolysis in MeOH, 78% yield)

¹H-NMR (500.15 MHz, CDCl₃): 0.78 (s, 3H), 0.86 (s, 3H), 0.98 (s, 3H), 1.24-1.29 (m, 1H), 1.36-1.41 (m, 1H), 1.44-1.50 (m, 1H) 1.52-1.62 (m, 3H), 1.63-1.70 (m, 1H), 1.75-1.80 (m 1H), 2.05-2.12 (m, 1H), 2.34 (td, 1H, J=13.4 Hz, J=7.1 Hz), 2.45-2.51 (m, 2H), 4.28 (dd, 1H, J=10.9 Hz, J=3.2 Hz), 4.51 (dd, 1H, J=10.9 Hz, J=8.4 Hz), 8.00 (s, 1H).

¹³C NMR (150 MHz, DMSO): δ 14.9, 18.3, 21.4, 23.1, 33.1, 33.1, 38.2, 41.1, 41.2, 41.2, 52.1, 57.8, 61.0, 162.0, 209.1.

### b) ((1S,4aS,8aS)-5,5,8a-trimethyl-2-oxodecahydronaphthalen-1-yl)methyl acetate (Ozonolysis in MeOH, 69% yield)

¹H-NMR (500.15 MHz, CDCl₃): 0.77 (s, 3H), 0.86 (s, 3H), 0.97 (s, 3H), 1.22-1.29 (m, 1H), 1.35-1.42 (m, 1H), 1.44-1.50 (m, 1H) 1.50-1.60 (m, 3H), 1.61-1.70 (m, 1H), 1.75-1.80 (m 1H), 2.00 (s, 3H), 2.04-2.10 (m, 1H), 2.35 (td, 1H, J=13.4 Hz, J=7.1 Hz), 2.41-2.50 (m, 2H), 4.18 (dd, 1H, J=11.0 Hz, J=3.2 Hz), 4.40 (dd, 1H, J=10.9 Hz, J=8.2 Hz).

¹³C NMR (100 MHz, CDCl₃): δ 15.4, 18.8, 21.1, 21.7, 23.8, 33.5, 33.6, 39.1, 41.8, 42.0, 42.2, 53.9, 58.9, 62.4, 171.1, 209.6.

### c) (1S,4aS,8aS)-5,5,8a-trimethyl-1-(((trimethylsilyl)oxy)methyl)octahydronaphthalen-2(1H)-one (Ozonolysis in EtOAc, 77% yield)

¹H-NMR (500.15 MHz, DMSO): 0.44 (s, 9H), 0.62 (s, 3H), 0.80 (s, 3H), 0.93 (s, 3H), 1.21-1.26 (m, 1H), 1.27-1.34 (m, 1H), 1.35-1.42 (m, 1H), 1.43-1.61 (m, 4H), 1.70-1.77 (m, 1H), 1.92-1.98 (m, 1H), 2.18-2.23 (m, 1H), 2.29-2.34 (m, 1H), 2.39-2.48 (m, 1H), 3.49 (dd, 1H, J=10.2 Hz, J=3.3 Hz), 3.88 (dd, 1H, J=10.3 Hz, J=7.0 Hz).

¹³C NMR (125 MHz, DMSO): δ −0.5, 14.9, 18.4, 21.4, 23.3, 33.1, 33.1, 38.4, 41.3, 41.4, 41.6, 52.5, 55.7, 65.1, 209.8.

### d) (1S,4aS,8aS)-1-((I-ethoxyethoxy)methyl)-5,5,8a-trimethyloctahydronaphthalen-2(1H)-one (Ozonolysis in EtOAc, 88% yield)

¹H-NMR (500.15 MHz, DMSO): 0.64 (s, 3H), 0.81 (s, 3H), 0.94 (s, 3H), 1.09 (td, 3H, J=7.1 Hz, J=2.7 Hz), 1.13 (dd, 3H, J=8.6 Hz, J=5.3 Hz), 1.19-1.33 (m, 2H), 1.36-1.42 (m, 1H), 1.43-1.61 (m, 4H), 1.67-1.75 (m, 1H), 1.92-2.00 (m, 1H), 2.19-2.25 (m, 1H), 2.34-2.40 (m, 1H), 2.40-2.49 (m, 1H), 3.49 (dd, 1H, J=10.2 Hz, J=3.3 Hz), 3.88 (dd, 1H, J=10.3 Hz, J=7.0 Hz), 3.29 (dd, 0.5H, J=9.6 Hz, J=3.3 Hz), 3.35-3.41 (m, 1H), 3.47 (dd, 0.5 H, J=9.6 Hz, J=3.4 Hz), 3.47-3.58 (m, 1H), 3.59 (dd, 0.5H, J=9.7 Hz, J=7.1 Hz), 3.83 (dd, 0.5H, J=9.5 Hz, J=6.9 Hz), 4.54-4.61 (m, 1H).

¹³C NMR (125 MHz, DMSO): δ 15.0, 15.2, 18.4, 19.8, 21.4, 23.3, 33.1, 33.1, 38.4, 41.3, 41.3, 41.4, 52.5, 59.1, 59.9, 62.7, 99.3, 209.7.

¹³C NMR (125 MHz, DMSO): δ 15.0, 15.1, 18.4, 19.6, 21.4, 23.3, 33.1, 33.1, 38.4, 41.2, 41.3, 41.4, 52.5, 59.1, 58.3, 62.7, 99.8, 209.6.

### e) (1S,4aS,8aS)-1-((benzyloxy)methyl)-5,5,8a-trimethyloctahydronaphthalen-2(1H)-one (Ozonolysis in MeOH, 78% yield)

¹H-NMR (500.15 MHz, CDCl₃): 0.72 (s, 3H), 0.84 (s, 3H), 0.96 (s, 3H), 1.21-1.29 (m, 1H), 1.32-1.40 (m, 1H), 1.40-1.60 (m, 4H), 1.60-1.70 (m, 1H), 1.70-1.76 (m 1H), 2.02-2.19 (m, 1H), 2.36 (tdd, 1H, J=13.1 Hz, J=7.0 Hz, J=0.7 Hz), 2.41-2.47 (m, 2H), 3.47 (dd, 1H, J=9.4 Hz, J=3.5 Hz), 3.91 (dd, 1H, J=9.5 Hz, J=6.8 Hz), 4.45 (d, 1H, J=11.9 Hz), 4.53 (d, 1H, J=11.9 Hz), 7.23-7.35 (m, 5H).

¹³C NMR (125 MHz, CDCl₃): δ 15.4, 18.9, 21.7, 23.9, 33.5, 33.6, 39.2, 41.9, 42.0, 42.4, 54.0, 64.1, 64.4, 73.4, 127.5, 127.7, 128.3, 138.6, 210.7.

### Example 14

### Preparation of (1S,4aS,8aS)-1-(hydroxymethyl)-5,5,8a-trimethyloctahydronaphthalen-2(1H)-one

A standard protocol using H₂SO₄ or KHCO₃ was used in most of the cases.
To 0.394 mmol of the starting material prepared in Example 13 in 400 mg MeOH were added 155 mg of a 5% aqueous solution of H₂SO₄ (0.2 eq). The mixture was stirred at room temperature until a full deprotection to (1S,4aS,8aS)-1-(hydroxymethyl)-5,5,8a-trimethyloctahydronaphthalen-2(1H)-one was observed (GC analysis using silylation). 753 mg of a 10% aqueous solution of K₂CO₃ (1.4 eq) were added and the MeOH was evaporated under reduced pressure. 5 g of cyclohexane were added and the mixture was washed twice with 5 g of water. The solvent was evaporated under reduced pressure (GC analysis using silylation).
or
To 0.394 mmol of the starting material prepared in Example 13 in 400 mg MeOH were added 8 mg of KHCO₃ was added (0.2 eq). The mixture was stirred at room temperature until a full deprotection to (1S,4aS,8aS)-1-(hydroxymethyl)-5,5,8a-trimethyloctahydronaphthalen-2(1H)-one was observed (GC analysis using silylation). The MeOH was evaporated under reduced pressure. 5 g of cyclohexane were added and the mixture was washed twice with 5 g of water. The solvent was evaporated under reduced pressure (GC analysis using silylation).

### a) ((1S,4aS,8aS)-5,5,8a-trimethyl-2-oxodecahydronaphthalen-1-yl)methyl formate to (1S,4aS,8aS)-1-(hydroxymethyl)-5,5,8a-trimethyloctahydronaphthalen-2(1H)-one

0.2 eq H₂SO₄ in water/MeOH, 4 h at room temperature, 100% conversion, 97.6% (1S,4aS,8aS)-1-(hydroxymethyl)-5,5,8a-trimethyloctahydronaphthalen-2(1H)-one by GC analysis (using silylation).
0.2 eq KHCO₃ in MeOH, 3.5 h at room temperature, 99% conversion, 97.4% (1S,4aS,8aS)-1-(hydroxymethyl)-5,5,8a-trimethyloctahydronaphthalen-2(1H)-one by GC analysis (using silylation).

### b) ((1S,4aS,8aS)-5,5,8a-trimethyl-2-oxodecahydronaphthalen-1-yl)methyl acetate to (1S,4aS,8aS)-1-(hydroxymethyl)-5,5,8a-trimethyloctahydronaphthalen-2(1H)-one and (1R,4aS,8aS)-1-(hydroxymethyl)-5,5,8a-trimethyloctahydronaphthalen-2(1H)-one

0.2 eq H₂SO₄ in water/MeOH, 3 d at room temperature, 99% conversion, 59.1% (1S,4aS,8aS)-1-(hydroxymethyl)-5,5,8a-trimethyloctahydronaphthalen-2(1H)-one and 15.7% of (1R,4aS,8aS)-1-(hydroxymethyl)-5,5,8a-trimethyloctahydronaphthalen-2(1H)-one by GC analysis (using silylation).

### c) (1S,4aS,8aS)-5,5,8a-trimethyl-1-(((trimethylsilyl)oxy)methyl)octahydronaphthalen-2(1H)-one to (1S,4aS,8aS)-1-(hydroxymethyl)-5,5,8a-trimethyloctahydronaphthalen-2(1H)-one

0.2 eq H₂SO₄ in water/MeOH, 2 h at room temperature, 100% conversion, 97.5% (1S,4aS,8aS)-1-(hydroxymethyl)-5,5,8a-trimethyloctahydronaphthalen-2(1H)-one by GC analysis (using silylation).

### d) (1S,4aS,8aS)-1-((i-ethoxyethoxy)methyl)-5,5,8a-trimethyloctahydronaphthalen-2(1H)-one to (1S,4aS,8aS)-1-(hydroxymethyl)-5,5,8a-trimethyloctahydronaphthalen-2(1H)-one

0.2 eq H₂SO₄ in water/MeOH, 2 h at room temperature, 99% conversion, 94.2% (1S,4aS,8aS)-1-(hydroxymethyl)-5,5,8a-trimethyloctahydronaphthalen-2(1H)-one by GC analysis (using silylation).

### e) (1S,4aS,8aS)-1-((benzyloxy)methyl)-5,5,8a-trimethyloctahydronaphthalen-2(1H)-one to (1S,4aS,8aS)-1-(hydroxymethyl)-5,5,8a-trimethyloctahydronaphthalen-2(1H)-one

300 mg (1S,4aS,8aS)-1-((benzyloxy)methyl)-5,5,8a-trimethyloctahydronaphthalen-2(1H)-one were reacted in the presence of 100 mg Palladium on characoal 10% in 10 mL EtOH in an autoclave (4 h at 20 psi H₂). A full conversion was observed (98% selectivity for 1S,4aS,8aS)-1-(hydroxymethyl)-5,5,8a-trimethyloctahydronaphthalen-2(1H)-one). The mixture was filtered on a pad of Celite® and the solvent was evaporated under reduced pressure.

### Example 15

### Preparation of (4aS,8aR)-5,5,8a-trimethyloctahydronaphthalen-2(1H)-one from (1S,4aS,8aS)-1-(hydroxymethyl)-5,5,8a-trimethyloctahydronaphthalen-2(1H)-one

An isolated Inox tube with heating system (Pyrolysis Oven, 3 cm×50 cm) was connected to an cooling condenser on the top of the column and to an evaporator system on the bottom. The column was heated up to 530° C. (internal) and the evaporator to 240° C. during 1 h. The whole system was set under vacuum (10 mbar). A mixture of 8.68 g (38.57 mmol) of (1S,4aS,8aS)-1-(hydroxymethyl)-5,5,8a-trimethyloctahydronaphthalen-2(1H)-one and 44 mL of 1-hexanol were added to the evaporator with a syringe pump (115 g/h) and the mixture was evaporated at 240° C. and distilled through the Inox tube (530° C. internal) and the product mixture (96% selectivity by GC) was collected with the cooling condenser in a flask. The evaporator and the column was purged with 1-hexanol (50 g) and heptane (90 g). After workup (saturated aqueous NaHCO₃ solution and water washes), solvent evaporation and distillation (4aS,8aR)-5,5,8a-trimethyloctahydronaphthalen-2(1H)-one was obtained with 97% selectivity (7.14 g, 36.75 mmol, 95% isolated yield)

### Example 16

### Preparation of (4aS,8aR)-5,5,8a-trimethyloctahydronaphthalen-2(1H)-one from (1S,4aS,8aS)-1-(hydroxymethyl)-5,5,8a-trimethyloctahydronaphthalen-2(1H)-one

### (Solvent Testing)

### General Protocol:

400 mg (95% purity, 1.69 mmol) (1S,4aS,8aS)-1-(hydroxymethyl)-5,5,8a-trimethyloctahydronaphthalen-2(1H)-one were dissolved in 6 mL of the solvent. The mixture was added slowly (26 mL/h) from the top (under a N₂ flow) to a heated pyrolysis column (23×2 cm, pyrolysis oven at 500° C.), which was filled with 56 g glass beads and was connected to a water cooled condenser and a 25 mL flask under the pyrolysis column. GC analysis of a drop of the mixture after 4 min gave the selectivity of the formation of (4aS,8aR)-5,5,8a-trimethyloctahydronaphthalen-2(1H)-one.
1-octanol: 89% product selectivity (GC), full conversion (GC)
4-methyl-2-pentanone: 81% product selectivity (GC), full conversion (GC)
1-hexanol: 87% product selectivity (GC), full conversion (GC)
1-butanol: 83% product selectivity (GC), full conversion (GC)
1-propanol: 92% product selectivity, full conversion (GC)
Ethanol: 89% product selectivity (GC), full conversion (GC)
Methanol: 83% product selectivity (GC after 14 min)
1-hexenal: 80% product selectivity (GC), full conversion (GC)
dibutylether: 84% product selectivity (GC), full conversion (GC)
cyclohexanone: 87% product selectivity (GC), full conversion (GC)
p-xylene: 87% product selectivity (GC), full conversion (GC)

### Example 17

### Preparation of (4aS,5S,8aS)-1,1,4a-trimethyl-6-methylene-5-vinyldecahydronaphthalene

The acetate (2-((1S,4aS,8aS)-5,5,8a-trimethyl-2-methylenedecahydronaphthalen-1-yl)ethyl acetate) or the tosylate (2-((1S,4aS,8aS)-5,5,8a-trimethyl-2-methylenedecahydronaphthalen-1-yl)ethyl 4-methylbenzenesulfonate) of 2-((1S,4aS,8aS)-5,5,8a-trimethyl-2-methylenedecahydronaphthalen-1-yl)ethan-1-ol can be prepared from (3aR,5aS,9aS,9bR)-3a,6,6,9a-tetramethyldecahydronaphtho[2,1-b]furan-2(1H)-one according the literature (G. Ohloff, W. Giersch Croatica Chem. Acta 1985, 58, 491-509). Elimination reaction of the acetate (thermal pyrolysis at 500° C.) or of the tosylate (DBU) gave (4aS,5S,8aS)-1,1,4a-trimethyl-6-methylene-5-vinyldecahydronaphthalene as a yellow liquid.

¹H-NMR (500.15 MHz, CDCl₃): 0.80 (s, 3H), 0.83 (s, 3H), 0.88 (s, 3H), 0.95-1.02 (m, 1H), 1.06-1.12 (m, 1H), 1.15-1.22 (m, 1H), 1.31-1.55 (m, 5H), 1.65-1.72 (m, 1H), 2.03-2.14 (m, 1H), 2.26-2.32 (m, 1H), 2.40-2.46 (m, 1H), 4.50-4.51 (m, 1H), 4.73-4.75 (m, 1H), 4.99 (dd, 1H, J=14.8 Hz, J=2.3 Hz), 5.09 (dd, 1H, J=10.2 Hz, J=2.5 Hz), 5.83 (dt, 1H, J=20.0 Hz, J=10.1 Hz).

¹³C NMR (125 MHz, CDCl₃): δ 14.9, 19.1, 21.9, 23.4, 33.5, 33.6, 36.8, 38.5, 40.6, 42.3, 54.7, 62.5, 107.7, 117.0, 136.7, 150.1.

Intermediates for the preparation of (4aS,5S,8aS)-1,1,4a-trimethyl-6-methylene-5-vinyldecahydronaphthalene:
(2-((1S,4aS,8aS)-5,5,8a-trimethyl-2-methylenedecahydronaphthalen-1-yl)ethyl acetate)

¹³C NMR (150 MHz, CDCl₃): δ 14.4, 19.3, 21.1, 21.7, 23.1, 24.3, 33.6, 33.6, 38.1, 39.0, 39.4, 42.1, 53.0, 55.5, 64.4, 106.6, 148.1, 171.2. (2-((1S,4aS,8aS)-5,5,8a-trimethyl-2-methylenedecahydronaphthalen-1-yl)ethyl 4-methylbenzenesulfonate)

¹³C NMR (150 MHz, CDCl₃): δ 14.3, 19.2, 21.6, 23.5, 24.2, 33.5, 33.6, 37.9, 38.8, 39.3, 42.0, 52.2, 55.3, 70.7, 106.4, 127.9, 129.8, 133.4, 144.5, 147.8.

### Example 18

### Preparation of (4aS,8aR)-5,5,8a-trimethyloctahydronaphthalen-2(1H)-one and (1R,4aS,8aS)-5,5,8a-trimethyl-2-oxodecahydronaphthalene-1-carboxylic acid from (4aS,5S,8aS)-1,1,4a-trimethyl-6-methylene-5-vinyldecahydronaphthalene

1 g (4.58 mmol) (4aS,5S,8aS)-1,1,4a-trimethyl-6-methylene-5-vinyldecahydronaphthalene were dissolved in 100 mL EtOAc in an ozonolysis reactor. The mixture was cooled to 0° C. and ozone was bubbled inside under stirring until a complete conversion of the starting material was observed by GC (KI test positive). After that oxygen (10 min) and nitrogen (10 min) were bubbled into the mixture. 4.26 g (16.03 mmol, 3.5 eq) PPh₃ were added. The cooling was removed after 1.5 h and the mixture was stirred overnight at room temperature (KI test was negative). The solvent was evaporated under reduced pressure (Rotavap in the fumehood). GC Analysis gave a 1/1 mixture of (4aS,8aR)-5,5,8a-trimethyloctahydronaphthalen-2(1H)-one and (4aS,8aS,Z)-1-(hydroxymethylene)-5,5,8a-trimethyloctahydronaphthalen-2(1H)-one. NMR analysis gave a mixture of (4aS,8aR)-5,5,8a-trimethyloctahydronaphthalen-2(1H)-one, (1R,4aS,8aS)-5,5,8a-trimethyl-2-oxodecahydronaphthalene-1-carboxylic acid and (4aS,8aS,Z)-1-(hydroxymethylene)-5,5,8a-trimethyloctahydronaphthalen-2(1H)-one.
This mixture could be fully transformed to (4aS,8aR)-5,5,8a-trimethyloctahydronaphthalen-2(1H)-one by an oxidative (CrO₃, acetone, heat) treatment (WO2012125488 Anderson, Eric; et al) or a basic (3 eq NaOAc in EtOH, reflux, 16 h, full conversion, selectivity >95%) treatment (decarbonylation/decarboxylation according to Xiang, H.; Zhao, Q.-L.; Xia, P.-J.; Xiao, J.-A.; Ye, Z.-P.; Xie, X.; Sheng, H.; Chen, X.-Q.; Yang, H. Org. Lett. 2018, 20, 1363-1366).
(4aS,8aS,Z)-1-(hydroxymethylene)-5,5,8a-trimethyloctahydronaphthalen-2(1H)-one
¹³C NMR (150 MHz, CDCl₃): 317.7, 18.9, 21.3, 25.0, 32.9, 33.1, 33.3, 35.3, 38.3, 41.4, 50.3, 122.8, 183.0, 188.9.
(4aS,8aS,Z)-1-(hydroxymethylene)-5,5,8a-trimethyloctahydronaphthalen-2(1H)-one could be also obtained by the oxidation with air (bubbling into the solution for 2 days at room temperature) of the intermediate of Example 6 ((((1S,4aS,8aS)-5,5,8a-trimethyloctahydro-1H-spiro[naphthalene-2,3′-[1,2,4]trioxolan]-1-yl)methanol)) in the presence of 20 mol % Fe(NO₃)₃·9H₂O, 20 mol % KCl and 20 mol % TEMPO. After the addition of PPh₃, aqueous workup/solvent evaporation (in the fume hood) and column chromatography (4aS,8aS,Z)-1-(hydroxymethylene)-5,5,8a-trimethyloctahydronaphthalen-2(1H)-one could be isolated in 67% yield over 2 steps (ozonolysis and oxidation). Smaller quantities (3% yield) of (4aS,8aR)-5,5,8a-trimethyloctahydronaphthalen-2(1H)-one were also isolated.

### Example 19

### Comparative Example

Benzyl (1R,4aS,8aS)-5,5,8a-trimethyl-2-oxodecahydronaphthalene-1-carboxylate could be prepared from (1S,4aS,8aS)-5,5,8a-trimethyl-2-methylenedecahydronaphthalene-1-carboxylic acid in 2 chemical steps (BzBr, K₂CO₃, Acetone and O₃, PPh₃, MeOH) or according to Pollini, G. P.; Bianchi, A.; Casolari, A.; Risi, C.; Zanirato, V.; Bertolasi, V. Tetrahedron: Asymmetry 2004, 15, 3223 from methyl (1R,4aS,8aS)-5,5,8a-trimethyl-2-oxodecahydronaphthalene-1-carboxylate.
Benzyl (1S,4aS,8aS)-5,5,8a-trimethyl-2-methylenedecahydronaphthalene-1-carboxylate

¹H-NMR (500.15 MHz, CDCl₃): 0.80 (s, 3H), 0.87 (s, 3H), 1.07 (s, 3H), 1.05-1.10 (m, 1H), 1.14-1.23 (m, 2H), 1.35-1.46 (m, 3H), 1.47-1-63 (m, 2H), 1.65-1.72 (m 1H), 2.00-2.10 (m, 1H), 2.36-2.44 (m, 1H), 2.86 (s, 1H), 4.65-4.67 (m, 1H), 4.80-4.83 (m, 1H), 5.10 (s, 2H), 7.28-7.37 (m, 5H).

¹³C NMR (150 MHz, CDCl₃): δ 14.3, 18.9, 21.7, 23.3, 33.4, 33.5, 36.3, 39.1, 39.4, 42.0, 54.6, 63.1, 65.7, 108.4, 128.1, 128.2, 128.5, 136.2, 143.7, 171.5.

Benzyl (1R,4aS,8aS)-5,5,8a-trimethyl-2-oxodecahydronaphthalene-1-carboxylate

¹³C NMR (125 MHz, CDCl₃): δ 14.9, 18.6, 21.7, 23.0, 33.5, 33.5, 39.1, 41.3, 41.8, 42.2, 53.2, 66.2, 70.0, 128.2, 128.2, 128.5, 135.8, 168.1, 205.4.

¹H-NMR analysis results in CDCl₃ were in accordance with data from literature (Pollini, G. P.; Bianchi, A.; Casolari, A.; Risi, C.; Zanirato, V.; Bertolasi, V. Tetrahedron: Asymmetry 2004, 15, 3223-3231).

Hydrogenation reaction (according to Pollini et al Tetrahedron: Asymmetry 2004, 15, 3223) of 108 mg Benzyl (1R,4aS,8aS)-5,5,8a-trimethyl-2-oxodecahydronaphthalene-1-carboxylate (33 mg Pd/characol10%, H₂, 2 mL EtOH, 2 h at 20 psi) led to the formation of (4aS,8aR)-5,5,8a-trimethyloctahydronaphthalen-2(1H)-one (selectivity 100%, full conversion). No trace of (1R,4aS,8aS)-5,5,8a-trimethyl-2-oxodecahydronaphthalene-1-carboxylic acid was observed (validated by silylation, methylation with trimethylsilyldiazomethane and NMR of the crude).

### Example 20

### Comparative Example

50 mg Methyl (1R,4aS,8aS)-5,5,8a-trimethyl-2-oxodecahydronaphthalene-1-carboxylate was heated in the presence of 10 mg KI in 0.5 mL DMF at reflux (150° C.) for 5 h (according to Ohloff, G.; Näf, E; Decorzant, R.; Thommen, W.; Sundt, E. Helv. Chim. Acta 1973, 56, 1414-1448). The formation of (4aS,8aR)-5,5,8a-trimethyloctahydronaphthalen-2(1H)-one was observed (full conversion of starting material). No trace of (1R,4aS,8aS)-5,5,8a-trimethyl-2-oxodecahydronaphthalene-1-carboxylic acid was detected (validated by silylation, methylation with trimethylsilyldiazomethane and NMR of the crude).

The invention claimed is:

1. A process for the preparation of a compound of formula 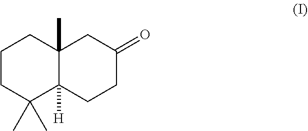
wherein the bold and hatched lines indicate a relative or absolute configuration comprising the steps of
a) oxidative cleavage of compound of formula 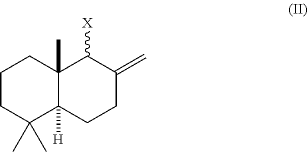
in a form of any one of its stereoisomers or a mixture thereof, and wherein X represents a vinyl, a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═O)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group; or one R^{c} and R^{d}, when taken together, form a C₄₋₅ oxacycloalkyl group;
into an intermediate of formula 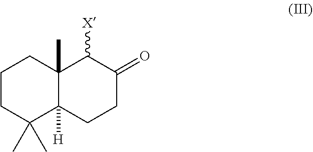
in the form of any one of its stereoisomers or a mixture thereof, and wherein X′ represents a CHO, a COOH, a C(═O)OR¹ or a CH₂OR² group and R¹ and R² have the same meaning as defined above; and
b) the conversion of the intermediate of formula (III) to the compound of formula (I).

2. The process according to claim 1, wherein the oxidative cleavage is an ozonolysis.

3. The process according to claim 1, wherein R¹ is a C₁₋₃ alkyl group and R² is a hydrogen atom or a C(═O)R^{a} group wherein R^{a} is a hydrogen atom or a C₁₋₃ alkyl group.

4. The process according to claim 1, wherein the conversion of the intermediate of formula (III) to the compound of formula (I) comprises a retro aldol reaction when X′ is CH₂OR².

5. The process according to claim 4, wherein a deprotection is carried out prior to the retro aldol reaction when R² is not a hydrogen atom.

6. The process according to claim 4, wherein the retro aldol reaction is a thermal retro aldol reaction.

7. The process according to claim 4, wherein the retro aldol reaction is carried out in a presence of a solvent having a boiling point equal to or greater than 65° C.

8. The process according to claim 1, wherein the conversion of the intermediate of formula (III) to the compound of formula (I) comprises a decarboxylation reaction when X′ is a COOH or a C(═O)OR¹ group.

9. The process according to claim 8, wherein the decarboxylation reaction is performed in the presence of an acid or a base.

10. The process according to claim 1, wherein X is a CH₂OH, a CHO or a COOH group and X′ is a CH₂OH or a COOH group.

11. The process according to claim 10, wherein the compound of formula (II) is obtained by contacting famesyl pyrophosphate with at least one enzyme.

12. The process according to claim 1, further comprising the step of converting compound of formula (I) to compound of formula 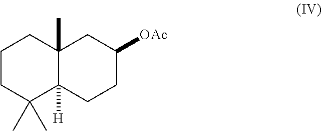 [SMILES: CC(=O)O[C@H]1CC[C@H]2C(C)(C)CCC[C@]2(C)C1]
wherein the bold and hatched lines indicate a relative or absolute configuration.

13. A compound of formula 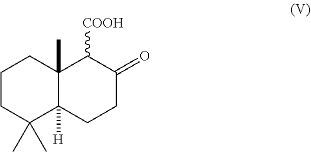 [SMILES: CC1(C)CCC[C@]2(C)C(C(=O)O)C(=O)CC[C@@H]12]
wherein the bold and hatched lines indicate a relative or absolute configuration.

14. A compound of formula 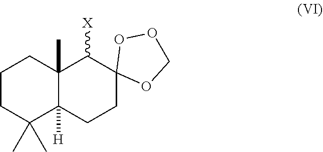
in a form of any one of its stereoisomers or a mixture thereof, and wherein the bold and hatched lines indicate a relative or absolute configuration; X represents a CHO, a COOH, a C(═O)OR¹ or a CH₂OR² group wherein R¹ represents a C₁₋₆ alkyl group and R² represents a hydrogen atom, a benzyl group, a C(═O)R^{a} group, a C(═O)OR^{b} group, a C(R^{c})₂OR^{d} group or a Si(R^{d})₃ group wherein R^{a} is a hydrogen atom or a C₁₋₆ alkyl group, R^{b} is a C₁₋₆ alkyl group, R^{c}, independently from each other, are a hydrogen atom or a C₁₋₂ alkyl group and R^{d} is a C₁₋₄ alkyl group or one R^{c} and R^{d}, when taken together, form a C₄₋₅ oxacycloalkyl group.

15. A compound of formula 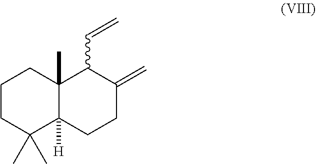 [SMILES: C=CC1C(=C)CC[C@H]2C(C)(C)CCC[C@]12C]
wherein the bold and hatched lines indicate a relative or absolute configuration.

16. The process according to claim 1, wherein R¹ is a methyl group.

17. The process according to claim 1, wherein R^{a} is a hydrogen atom.

18. The process according to claim 4, wherein the retro aldol reaction is carried out in a presence of a solvent having a boiling point equal to or greater than 110° C.

19. The process according to claim 1, wherein X is a CH₂OH group and X′ is a CH₂OH group.
